(19) United States
(12) Patent Application Publication
(10) Pub. No.: US 2017/0081686 A1
(43) Pub. Date: Mar. 23, 2017

(54) MODIFIED METHANOTROPHIC BACTERIA AND USES THEREOF

(71) Applicant: UNIVERSITY OF WASHINGTON, Seattle, WA (US)

(72) Inventors: Mary Lidstrom, Seattle, WA (US); Marina Kalyuzhnaya, Seattle, WA (US); Valentina Khmelenina, Seattle, WA (US); Sergey But, Seattle, WA (US)

(73) Assignee: UNIVERSITY OF WASHINGTON, Seattle, WA (US)

(21) Appl. No.: 15/311,375

(22) PCT Filed: May 15, 2015
(86) PCT No.: PCT/US15/31088

§ 371 Date: Nov. 15, 2016

Related U.S. Application Data
(60) Provisional application No. 61/993,636, filed on May 15, 2014.

(51) Int. Cl. C12P 19/02; C12M 1/34; C12P 19/16; C12N 15/52; C12M 1/00; C12M 1/107; C12N 1/30

(52) U.S. Cl. CPC C12P 19/02; C12M 21/04; C12M 41/12; C12M 41/34; C12M 41/40; C12N 1/30; C12N 15/52; C12M 41/26; C12M 47/10; C12M 29/12; C12P 19/16

(57) ABSTRACT

Described herein are compositions and methods relating to the bacterial production of industrially-useful carbon products from methane. In particular, the engineered bacteria described herein have been modified to increase the production sucrose. Aerobic methanotrophic bacteria (methanotrophs or MB) are a highly specialized group of microbes utilizing methane (e.g., CH4) as a sole source of carbon and energy. Methanotrophic bacteria function in nature by eliminating methane and retaining it in the carbon cycle. The biotechnological potential of MB has been of broad interest, ranging from bioremediation to large scale bacterial protein production.

### CROSS-REFERENCE TO RELATED APPLICATIONS

This application claims benefit under 35 U.S.C. §119(e) of U.S. Provisional Application No. 61/993,636 filed May 15, 2014, the contents of which are incorporated herein by reference in their entirety.

### STATEMENT REGARDING FEDERALLY SPONSORED RESEARCH

This invention was made with U.S. government support under MCB-0842686, awarded by the National Science Foundation. The U.S. Government has certain rights in the invention.

### FIELD OF THE INVENTION

The field of the invention relates to the modification of methanotrophic bacteria and uses thereof.

### BACKGROUND

Methane is a critical component of Earth's carbon cycle and contributes up to 25% of the global warming (Forster&Gregory, 2006; Wuebbler&Hayhoe. 2002). CH₄ is emitted from a variety of natural and anthropogenic sources (EPA 2008, 2010; Scheehle et a., 2001; Scheutz et al, 2009; Shindell et al., 2012; World Bank, 2008). Human-related activities, such as fossil fuel production (e.g., underground coal mining, oil and gas production), agriculture (e.g., enteric fermentation in livestock, manure management, and rice cultivation), landfills, and municipal wastewater are major contributors to global CH₄ emission. New sources of methane emission (up to 4% of global emission) include biofuel production (Piccot & Sridhar 1996; Schahczenski & Hill, 2009; Aydin et al., 2011; Fargione et al., 2010). Anthropogenic CH₄ emission accounts for more than 60% of the total CH₄ budget (≈300 tg yr −1) (EPA 2008, 2010; Shindell et al., 2012). As the understanding of global climate change has increased, a multitude of research activities have been directed towards reducing methane emission and optimizing low-cost methane mitigation technologies.

Many bio-refineries produce methane as a byproduct of biomass-waste treatment. Anaerobic digestions are the most common way of handling residual waste and biomass. This biological platform has gained popularity due to the relatively simple operation, installation and flexibility that enables both small and large-scale biogas production. The typical bulk biogas composition is: 45-60% CH₄; 35-55% CO₂ and up to 6% of H₂, H₂S, water, volatile organic carbons (VOC), and N₂O. Low energy density (80 Btu/gallon), low heat and electric conversion efficiencies (25-30%), seasonal fluctuations, costly equipment for compression and H₂5 scrubbing (Hullu et al., 2008), and its highly corrosive nature limit utilization of biogas as energy carrier. As a result this renewable gas is mostly flared (Krich et al., 2005; Cuellar et al., 2008). Similar to natural gas, the biogas flares contribute to local air pollution (emission of SO₂, CO, amines, siloxanes, mercaptens, NOx and formaldehyde) (EPA, 2008). Implementation of a biological methane scrubbing technology has a great potential for mitigation of these harmful effects, due to the high specificity and high efficiency of microbial systems (Scheutz et al., 2009). Biotechnology based on production of value-added chemicals from low grade biogas can dramatically improve sustainability of waste treatment, livestock, and biofuel facilities.

### SUMMARY

Aerobic methanotrophic bacteria (methanotrophs or MB) are a highly specialized group of microbes utilizing methane (e.g., CH₄) as a sole source of carbon and energy. Methanotrophic bacteria function in nature by eliminating methane and retaining it in the carbon cycle. The biotechnological potential of MB has been of broad interest, ranging from bioremediation to large scale bacterial protein production. The current disclosure provides modified MB that produce sucrose from methane including, for example, wasted/flared biogas, as a way to improve sustainability of biomass-producing plant and natural gas, as a way to substitute expensive agriculture-dependent production with a cheap, pipeline source of sucrose. The modified MB described herein have the catalytic versatility to produce extractable sugars from methane.

In one aspect, the methods and compositions described herein relate to an engineered methanotrophic bacterium, the bacterium comprising at least one genetic alteration that increases flux through the sucrose biosynthesis pathway, thereby stimulating the conversion of methane to sucrose.

In one embodiment of this aspect and all other aspects described herein, the genetic alteration results in an increase in the activity of at least one of the metabolic enzymes selected from the group consisting of: methane monoxygenase, hexulose phosphate synthase, phospho-3-hexuloisomerase, and sucrose-phosphate synthase.

In another embodiment of this aspect and all other aspects described herein, the genetic alteration includes overexpression of at least one of the metabolic enzymes selected from the group consisting of: methane monoxygenase, hexulose phosphate synthase, phospho-3-hexuloisomerase, and sucrose-phosphate synthase.

In another embodiment of this aspect and all other aspects described herein, the genetic alteration results in a decrease in the activity of at least one of the metabolic enzymes selected from the group consisting of: amylase, and glucose-1-phosphate adenyltransferase.

In another embodiment of this aspect and all other aspects described herein, the genetic alteration is a mutation in the gene encoding at least one of the metabolic enzymes selected from the group consisting of: amylase, and glucose-1-phosphate adenyltransferase.

In another embodiment of this aspect and all other aspects described herein, the bacterium further comprises at least one additional genetic alteration that (i) reduces the activity of at least one of the metabolic enzymes selected from the group consisting of: gluconate-6-phosphate dehydrogenase, ADP-glucose pyrophosphorylase, glycogen synthase, and glycogen branching enzyme, and/or (ii) increases the activity of methane monoxygenase.

In another embodiment of this aspect and all other aspects described herein, the bacterium comprises between 0.1%-30% sucrose content.

In another embodiment of this aspect and all other aspects described herein, the sucrose content or production is increased by at least 20% as compared to the corresponding wild-type bacterium exposed to the same conditions.

In another embodiment of this aspect and all other aspects described herein, the bacterium is selected from the group consisting of: 20Z, 5GB1, Methylobacter bovis, Methylomonas sp. LW13, Methylomonas MK1, Methylomicorbium buryatense 5G, Metholobacter luteus. Methylocaldum szegediense, Methylobacter marinus, Methylobactertundripaludum, Methyloglobulus morosus KoM1, Methylohalobius crimeensis, Methylomicrobium album, Methylomonas methanica, Methylosarcina fibrate, Methylosarcina lacus LW14, Methylobacter sp. 31-32, Methylovulum miyakonense strain HT12, Methylococcus capsulatus Bath, Methylococcus capsulatus Texas, and Methylomonas sp.11b.

In another embodiment of this aspect and all other aspects described herein, the bacterium further comprises an additional genetic alteration that reduces flux through the ectoine biosynthesis pathway.

In another embodiment of this aspect and all other aspects described herein, the genetic alteration results in a decrease in the activity of at least one of the metabolic enzymes selected from the group consisting of: ect A, ectB, and ectC.

In another embodiment of this aspect and all other aspects described herein, the bacterium comprises a carbon conversion efficiency of at least 45%.

In another embodiment of this aspect and all other aspects described herein, the bacterium produces at least 0.1 g sucrose/L fermentation broth/hr.

Another aspect described herein relates to a method for fixing methane carbon in sucrose, the method comprising contacting a genetically modified bacterium as described herein with a gaseous substrate comprising methane, under conditions suitable for methane catabolism.

Also provided herein, in another aspect, is a method for performing dry fermentation of methane to sucrose, the method comprising: contacting bacteria as described herein with a gaseous mixture comprising methane in the absence of exogenously applied water, thereby dry fermenting methane to sucrose.

In one embodiment of this aspect and all other aspects described herein, the method further comprises a step of removing metabolic water and/or sucrose produced by the bacteria during dry fermentation.

In another embodiment of this aspect and all other aspects described herein, the bacteria are immobilized on a solid support.

In another embodiment of this aspect and all other aspects described herein, the solid support comprises a filter.

In another embodiment of this aspect and all other aspects described herein, the solid support comprises a polymer.

In another embodiment of this aspect and all other aspects described herein, the method further comprises a step of introducing additional bacteria having a genetic modification as described herein.

Another aspect described herein relates to a method of removing methane from a gaseous waste stream comprising contacting a gaseous waste stream comprising methane with bacteria as described herein.

Also provided herein, in another aspect, is a methane fermentation bioreactor, comprising a plurality of solid supports comprising immobilized, viable, methanotrophic bacteria according to claim 1, the supports located in a chamber comprising a first inlet supplying a mixture of methane and air or oxygen, and a second inlet permitting periodic flushing of the solid supports with an aqueous composition to remove sucrose produced by the bacteria and a first outlet permitting collection of sucrose, wherein said supports are arranged and held in the gas phase during methane fermentation, and wherein the bacteria remain viable and metabolically active for fermentation using water they produce via methane fermentation, exogenous water not being necessary for viability or metabolic activity.

In one embodiment of this aspect and all other aspects described herein, the bioreactor further comprises a second outlet designed and arranged in conjunction with the first inlet to permit a flow of methane and air or oxygen over or through the supports during methane fermentation.

In another embodiment of this aspect and all other aspects described herein, the bioreactor maintains a temperature between 15-37° C.

In another embodiment of this aspect and all other aspects described herein, the bioreactor maintains a humidity of at least 30%.

In another embodiment of this aspect and all other aspects described herein, the bioreactor comprises one or more of temperature, pressure, humidity and gas flow rate sensors. In another embodiment of this and all other aspects described herein, the bioreactor comprises one or more modules controlling temperature, pressure and humidity in the reactor as well as flow rate of the gaseous substrate.

In another embodiment of this aspect and all other aspects described herein, the methanotrophic bacteria further comprise a genetic alteration to enhance sucrose production using methane as a carbon source.

In another embodiment of this aspect and all other aspects described herein, the genetic alteration results in an increase in the activity of at least one of the metabolic enzymes selected from the group consisting of: methane monoxygenase, hexulose phosphate synthase, phospho-3-hexuloisomerase, and sucrose-phosphate synthase.

In another embodiment of this aspect and all other aspects described herein, the genetic alteration is overexpression of at least one of the metabolic enzymes selected from the group consisting of: methane monoxygenase, hexulose phosphate synthase, phospho-3-hexuloisomerase, and sucrose-phosphate synthase.

In another embodiment of this aspect and all other aspects described herein, the genetic alteration results in a decrease in the activity of at least one of the metabolic enzymes selected from the group consisting of: amylase, and glucose-1-phosphate adenyltransferase.

In another embodiment of this aspect and all other aspects described herein, the genetic alteration is a mutation in the gene encoding at least one of the metabolic enzymes selected from the group consisting of: amylase, and glucose-1-phosphate adenyltransferase.

In another embodiment of this aspect and all other aspects described herein, the bioreactor further comprises at least one additional genetic alteration that (i) reduces the activity of at least one of the metabolic enzymes selected from the group consisting of: gluconate-6-phosphate dehydrogenase, ADP-glucose pyrophosphorylase, glycogen synthase, and glycogen branching enzyme, and/or (ii) increases the activity of methane monoxygenase.

In another embodiment of this aspect and all other aspects described herein, the bacteria comprise between 0.1%-30% sucrose content.

In another embodiment of this aspect and all other aspects described herein, the sucrose content is increased by at least 20% as compared to the corresponding wild-type bacterium exposed to the same conditions.

In another embodiment of this aspect and all other aspects described herein, the methanotrophic bacteria is selected from the group consisting of: 20Z, 5GB1, Methylobacter bovis, Methylomonas sp. LW13, Methylomonas MK1, Methylomicorbium buryatense 5G, Metholobacter luteus. Methylocaldum szegediense, Methylobacter marinus, Methylobactertundripaludum, Methyloglobulus morosus KoM1, Methylohalobius crimeensis, Methylomicrobium album, Methylomonas methanica, Methylosarcina fibrate, Methylosarcina lacus LW14, Methylobacter sp. 31-32, Methylovulum miyakonense strain HT12, Methylococcus capsulatus Bath, Methylococcus capsulatus Texas, and Methylomonas sp.11b.

In another embodiment of this aspect and all other aspects described herein, the methanotrophic bacteria further comprise an additional genetic alteration that reduces flux through the ectoine biosynthesis pathway.

In another embodiment of this aspect and all other aspects described herein, the methanotrophic bacteria further comprise a genetic alteration that results in a decrease in the activity of at least one of the metabolic enzymes selected from the group consisting of: ect A, ectB, and ectC.

In another embodiment of this aspect and all other aspects described herein, the methanotrophic bacteria comprise a carbon conversion efficiency of at least 45%.

In another embodiment of this aspect and all other aspects described herein, the methanotrophic bacteria produces at least 0.1 g sucrose/L fermentation broth/hr.

In another embodiment of this aspect and all other aspects described herein, the solid support comprises a filter.

In another embodiment of this aspect and all other aspects described herein, the solid support comprises a polymer.

### BRIEF DESCRIPTION OF THE FIGURES

FIG. 1 Overview of the sucrose metabolism of Methylomicrobium (M.) acaliphilum 20Z. SPS, sucrose-phosphate synthase; SPP, sucrose-phosphate phosphatase; FruK, fructokinase; Ams, amylosucrose; GS, glycogen synthase; MMO, methane monooxygenase; MDH, methanol dehydrogenase; FAOx-pathways, formaldehyde oxidation pathways. FDH, formate dehydrogenase; HPS, hexulose-phosphate synthase; HPI, hexulose-phosphate isomerase; PGI, phosphoglucose isomerase; PGM, phosphoglucomutase; UGP, UDP-glucopyrophosphorylase; AGP, ADP-glucopyrophosphorylase; GS—glycogen synthase.

FIG. 2 Sucrose accumulation in the cells of the M. acaliphilum strain 20Z (wild type), ΔectBC, Δams, M. acaliphilum strain 20ZER lacking amylsucrose; strain lacking ectBC genes; and 20ZΔsps::sps, the strain harboring pGM-Pect:sps plasmid (overexpression of Sps).

FIG. 3 20ZR glycogen biosynthesis (glgAB1, glgAB2) genes (deleted region in modified form of strain underlined); as used herein, “R” signifies rifamycin resistance as compared to wild-type strains.

FIG. 4 20ZR, ectBC, (ectBC) gene (deleted region in modified form of strain underlined).

FIG. 5 20ZR, Alpha amylase (ams) gene (deleted region in modified form of strain underlined).

FIGS. 6A-6B The membrane module with immobilized methanotrophic cells. FIG. 6A. System used for methane consumption studies by immobilized semi-dry cells. FIG. 6B. A prototype of lab-scale module for evaluation of methane and oxygen consumption and sucrose excretion. FUs-filter units.

### DETAILED DESCRIPTION

Described herein are compositions and methods relating to the bacterial production of industrially-useful carbon products from methane. In particular, the engineered bacteria described herein have been modified to increase the production sucrose. Such bacteria can be used to convert waste methane to sucrose, which can be used in a variety of applications. In some embodiments, the bacteria are used under dry conditions to fix methane in the form of sucrose, a process referred to herein as “dry fermentation.”

### DEFINITIONS

As used herein the term “carbon flux” or “flux” refers to the number of feedstock molecules (e.g., methane) which proceed down the desired pathway and/or are incorporated into a target molecule relative to competitive paths and/or molecules per unit time. Thus, increased sucrose flux refers to an increase in the amount of carbon (e.g., from methane) that is converted into sucrose over a specific time period (e.g., per minute).

The term “gaseous substrate” includes any gas which contains a compound or element used by a microorganism as a carbon source and optionally energy source in microbial conversion. The gaseous substrate will typically contain a significant proportion of CH₄ and air and/or O₂. Similarly, the term “substrate” includes any gas and/or liquid which contains a compound or element used by a microorganism as a carbon source and optionally energy source in microbial conversion. Examples of liquid substrates include methanol. Examples of gaseous substrates include methane, as well as all C1 carbon substrates such as any carbon-containing molecule that lacks a carbon-carbon bond such as methanol, formaldehyde, formic acid, formate, methylated amines (e.g., mono-, di-, and tri-methyl amine), methylated thiols, and carbon dioxide.

As used herein, “methanotrophic bacteria” are bacteria that are able to metabolize methane as their primary source of carbon. In certain embodiments of the present disclosure, methanotrophic bacteria include, but are not limited to, Methylococcus, Methylomonas, Methylomicrobium, Methylobacter, Methylocaldum, Methylovulum, Methylomarinum, Methylocystis and Methylosinus. In other embodiments, methanotrophic bacteria include, but are not limited to, Methylomicrobium buryatense and Methylomicrobium alcaliphilum. In additional embodiments, the methanotrophic bacteria are Methylomicrobium buryatense 5GB1 and Methylomicrobium alcaliphilum 20Z.

As used herein, “engineered” refers to the aspect of having been manipulated by the hand of man. For example, in some embodiments of methods and compositions described herein, an engineered bacterium comprises an engineered polynucleotide, e.g., comprises a genetic alteration resulting in a polynucleotide sequence, copy number, or regulatory element not found in nature. As is common practice and is understood by those in the art, progeny and copies of an engineered polynucleotide are typically still referred to as “engineered” even though the actual manipulation was performed on a prior entity. As used herein, “genetic alteration” refers to a change or difference in the genetic material of a cell as compared to a reference wildtype cell, e.g., a deletion, an insertion, a SNP, a substitution, a gene rearrangement, a mutation, and/or the introduction of an exogenous gene or sequence. In some embodiments, the genetic alteration can be an engineered change.

As used herein, “modulation” with respect to genes, proteins, reactions, and/or pathways, refers to downregulation (inhibits activity) or upregulation (activates or increases activity) of protein activity or function. In one embodiment, the modulation occurs by directly inhibiting or increasing the activity of a protein, i.e., via direct physical interaction with the protein or a nucleic acid encoding the protein. In one embodiment, the activity of the protein is modulated indirectly, for example, in signaling, by inhibiting an upstream effector of the protein activity. In some embodiments, the activity of the protein is modulated by increasing or decreasing the level of the protein, e.g., by increasing or decreasing the expression of the gene encoding the protein. In some embodiments of this and other aspects of the technology described herein, activity of the protein is inhibited or lowered by at least 5%, at least 10%, at least 20%, at least 30%, at least 40%, at least 50%, at least 60%, at least 70%, at least 80%, at least 90%, at least 95%, at least 98%, or even 100% (i.e., complete loss of activity) relative to an uninhibited control. In some embodiments of this and other aspects of the technology described herein, activity of the protein is increased by at least 5%, at least 10%, at least 20%, at least 30%, at least 40%, at least 50%, at least 60%, at least 70%, at least 80%, at least 90%, at least 1-fold, at least 1.1-fold, at least 1.5-fold, at least 2-fold, at least 3-fold, at least 4-fold, at least 5-fold, or more relative to an un-activated control, e.g., in absence of activating agent.

A genetic alteration resulting in a decrease in the activity and/or level of a target gene/protein can include, e.g., a knock-down (e.g., a mutation in a promoter that results in decreased gene expression), a knock-out (e.g., a mutation or deletion that results in 99% or greater decrease in gene expression or activity), a mutation of catalytic residues that reduces enzymatic activity, and/or introduction of a nucleic acid sequence that reduces the expression of the target gene (e.g., a repressor that inhibits expression of the target or inhibitory nucleic acids using e.g., CRISPR etc.).

A genetic alteration resulting in the increase of the activity and/or level of a target gene/protein can include, e.g., introduction of an exogenous nucleic acid sequence comprising the target gene (e.g., on a plasmid or integrated into the genome), a mutation of the endogenous target gene to increase expression (e.g., a mutation of the promoter sequence), and/or introduction of a nucleic acid sequence that increases the expression of the target gene (e.g., introduction of a transcription factor that increases expression of the target gene). In some embodiments, any of these changes can result in ectopic expression of a polypeptide.

In some embodiments, an engineered methanotrophic bacterium as described herein comprises a genetic alteration causing an increase in the conversion of methane to sucrose.

In some embodiments, a heterolog, homolog, and/or variant of the genes described can be utilized in the methods and compositions described herein. A “variant,” as referred to herein, is a polypeptide substantially homologous to a native or reference polypeptide, but which has an amino acid sequence different from that of the native or reference polypeptide because of one or a plurality of deletions, insertions or substitutions. Such polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of ordinary skill in the art will recognize that individual substitutions, deletions or additions to a nucleic acid, peptide, polypeptide, or protein sequence which alter a single amino acid or a small percentage, (i.e. 5% or fewer, e.g., 4% or fewer, or 3% or fewer, or 1% or fewer) of amino acids in the encoded sequence is a “conservatively modified variant” where the alteration or alterations result in the substitution of an amino acid with a chemically similar amino acid. It is contemplated that some changes can potentially improve the relevant activity, such that a variant, whether conservative or not, has more than 100% of the activity of a wildtype or native polypeptide, e.g., 110%, 125%, 150%, 175%, 200%, 500%, 1000% or more.

Amino acid sequence alignment of a polypeptide of interest with a reference, e.g., from another species can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Such alignments are readily created by one of ordinary skill in the art, e.g., using the default settings of the alignment tool of the BLASTP program. Furthermore, homologs of any given polypeptide or nucleic acid sequence can be found using BLAST programs, e.g., by searching freely available databases of sequence for homologous sequences, or by querying those databases for annotations indicating a homolog (e.g., search strings that comprise a gene name or describe the activity of a gene).

The variant amino acid or DNA sequence can be at least 90%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g., BLASTp (available on the world wide web), with default parameters set.

In some embodiments, the variant is a conservative substitution variant. Variants can be obtained by mutations of native nucleotide sequences, for example.

A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g., 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype protein (e.g., enzyme), e.g., 110%, 125%, 150%, 175%, 200%, 500%, 1000% or more.

A given amino acid can be replaced by a residue having similar physiochemical characteristics referred to herein as a “conservative mutation”, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained, or for that matter, improved upon. Conservative substitution tables providing functionally similar amino acids are well known in the art. Such conservatively modified variants are in addition to and do not exclude polymorphic variants, interspecies homologs, and alleles consistent with this disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

A methanotrophic bacterium which is engineered as described herein can be, e.g., a Methylomicrobium spp.; Methylmonas spp.; Group I methanotrophic bacterium; Methylomicrobium alcaliphilum; M. alcaliphilum 20ZR; M. buryatenase; M. buryatenase 5GB1; Methylomonas sp. LW13; Methylmonas MK1; or Methylomonas sp.11b.

As described herein, an “inducible promoter” is one that is characterized by initiating or enhancing transcriptional activity when in the presence of, influenced by, or contacted by an inducer or inducing agent relative to such activity when not in the presence of, under the influence of, or in contact with the inducer or inducing agent. An “inducer” or “inducing agent” can be endogenous, or a normally exogenous compound or protein that is administered in such a way as to be active in inducing transcriptional activity from the inducible promoter. In some embodiments, the inducer or inducing agent, e.g., a chemical, a compound or a protein, can itself be the result of transcription or expression of a nucleic acid sequence (e.g., an inducer can be a transcriptional repressor protein), which itself may be under the control or an inducible promoter. Non-limiting examples of inducible promoters include but are not limited to, the lac operon promoter, a nitrogen-sensitive promoter, an IPTG-inducible promoter, a salt-inducible promoter, and tetracycline, steroid-responsive promoters, rapamycin responsive promoters and the like. Inducible promoters for use in prokaryotic systems are well known in the art, see, e.g., the beta lactamase and lactose promoter systems, the arabinose promoter system, including the araBAD promoter, the rhamnose promoter, the alkaline phosphatase promoter, a tryptophan (trp) promoter system, the PLtetO-1 and Plac/are-1 promoters, and hybrid promoters such as the tac promoter.

An inducible promoter useful in the methods and systems as disclosed herein can be induced by one or more physiological conditions, such as changes in pH, temperature, radiation, osmotic pressure, saline gradients, cell surface binding, and the concentration of one or more extrinsic or intrinsic inducing agents. The extrinsic inducer or inducing agent can comprise amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones, and combinations thereof. In specific embodiments, the inducible promoter is activated or repressed in response to a change of an environmental condition, such as the change in concentration of a chemical, metal, temperature, radiation, nutrient or change in pH. Thus, an inducible promoter useful in the methods and systems as disclosed herein can be a phage inducible promoter, nutrient inducible promoter, temperature inducible promoter, radiation inducible promoter, metal inducible promoter, hormone inducible promoter, steroid inducible promoter, and/or hybrids and combinations thereof. Appropriate environmental inducers can include, but are not limited to, exposure to heat (i.e., thermal pulses or constant heat exposure), various steroidal compounds, divalent cations (including Cu2+ and Zn2+), galactose, tetracycline, IPTG (isopropyl-(3-D thiogalactoside), as well as other naturally occurring and synthetic inducing agents and gratuitous inducers.

Inducible promoters useful in the methods and systems as disclosed herein also include those that are repressed by “transcriptional repressors” that are subject to inactivation by the action of environmental, external agents, or the product of another gene. Such inducible promoters may also be termed “repressible promoters” where it is required to distinguish between other types of promoters in a given module or component of the biological switch converters described herein. Preferred repressors for use in the present invention are sensitive to inactivation by physiologically benign agent. Thus, where a lac repressor protein is used to control the expression of a promoter sequence that has been engineered to contain a lacO operator sequence, treatment of the host cell with IPTG will cause the dissociation of the lac repressor from the engineered promoter containing a lacO operator sequence and permit transcription to occur. Similarly, where a tet repressor is used to control the expression of a promoter sequence that has been engineered to contain a tetO operator sequence, treatment of the host cell with tetracycline will cause the dissociation of the tet repressor from the engineered promoter and permit transcription of the sequence downstream of the engineered promoter to occur.

In some embodiments, a genetic alteration is present in nucleic acid sequence present within the prokaryotic genome, e.g., the nucleic acids can be incorporated into the genome. For example, in bacteria, one can use homologous recombination to target genes to specific sites on bacterial chromosomes. In some embodiments, a nucleic acid comprising a genetic alteration is present within a vector. The term “vector”, as used herein, refers to a nucleic acid construct designed for delivery to a host cell or transfer between different host cells. As used herein, a vector can be viral or non-viral. Many vectors useful for transferring exogenous genes into target cells are available, e.g., the vectors may be episomal, e.g., plasmids, virus derived vectors or may be integrated into the target cell genome, through homologous recombination or random integration. In some embodiments, a vector can be an expression vector. As used herein, the term “expression vector” refers to a vector that has the ability to incorporate and express heterologous nucleic acid fragments in a cell. An expression vector may comprise additional elements. The nucleic acid incorporated into the vector can be operatively linked to an expression control sequence when the expression control sequence controls and regulates the transcription and translation of that polynucleotide sequence.

In some embodiments, a nucleic acid comprising a genetic alteration is present within a portion of a plasmid. Plasmid vectors can include, but are not limited to, pBR322, pBR325, pACYC177, pACYC184, pUC8, pUC9, pUC18, pUC19, pLG339, pR290, pKC37, pKC101, SV 40, pBluescript II SK+/− or KS+/−, pQE, pIH821, pGEX, pET series. Other vectors useful for introducing modifications to or manipulating methanotrophic bacteria are known to those of skill in the art.

The term “exogenous” refers to a substance present in a cell other than its native source. The term “exogenous” when used herein can refer to a nucleic acid or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in low amounts and one wishes to increase the amount of the nucleic acid or polypeptide in the cell or organism. A substance will be considered exogenous if it is introduced into a cell or an ancestor of the cell from which the cell has inherited the substance. In contrast, the term “endogenous” refers to a substance that is native to the biological system or cell (e.g., the microbial cell and/or target cell). As used herein, “ectopic” refers to a substance that is found in an unusual location and/or amount. An ectopic substance can be one that is normally found in a given cell, but at a much lower amount and/or at a different time.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA (e.g., genomic DNA or cDNA). In another aspect, the nucleic acid can be RNA, including mRNA.

As used herein, “donor bacteria” are bacteria containing genetic material which is capable of being transferred to other bacteria. Genetic material from donor bacteria can be transferred, e.g., to recipient bacteria.

As used herein, “recipient bacteria” are bacteria that receive or are capable of accepting genetic material from another source, e.g., another bacterium.

As used herein, “broad host range plasmid” is a plasmid capable of replicating in more than one bacterial host. In certain embodiments, the broad host range plasmid can replicate in 2, 3, 4, 5, 6 or more different types or species of hosts. See, for example, Lale et al., Methods in Molecular Biology, vol. 765, pages 327-343, (2011).

As used herein, “conditions suitable for dry fermentation” refers to dry conditions under which a detectable level of fermentation occurs. Such conditions can comprise those under which a bacterium as described herein is metabolically active and provided access to methane. Examples of suitable conditions are provided, e.g., in the Examples herein. In one embodiment of the methods described herein, the conditions suitable for dry fermentation are aerobic conditions. Alternatively, dry fermentation can be performed under conditions of restricted aerobic conditions (e.g., conditions wherein aerobic respiration does not account for 100% of energy production, often when oxygen levels are present but below those observed in ambient air (e.g., less than 21% O₂) or anaerobic conditions (e.g., in the absence of oxygen). For the avoidance of doubt, “dry fermentation” and “conditions suitable for dry fermentation” refer to fermentation of methane which does not require the addition of water. In some embodiments, methanotrophic bacteria engineered as described herein produce sufficient water (e.g., metabolic water) upon catabolism of methane to survive without added water. Thus, “dry fermentation” as the term is used herein refers to fermentation in which methanotrophic bacteria provided methane as a carbon and energy source produce sufficient water to remain viable without added water. It is noted that water may be used to harvest or remove sucrose produced by the engineered bacteria, and this harvest or removal may be performed cyclically, but that the bacteria are not maintained or suspended or immersed in water or aqueous medium during the fermentation period between sucrose collection cycles. Preferred humidity for dry fermentation is 30% or greater and under these conditions some embodiments of the engineered methanotrophic bacteria as described herein, when supplied with gaseous methane, will remain viable and active for fermentation of methane to sucrose for an extended period (e.g., one month, two months, three months or more).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

### Methanotrophic Bacteria (MB)

Methanotrophs are a highly specialized bacterial group utilizing methane (e.g., CH₄) as a sole source of carbon and energy. Obligate aerobic MB can be separated into three major groups. Group I MB are gammaproteobacteria that have stacked membranes built mostly of C16 fatty acids. Group I MB use the ribulose monophosphate (RuMP) cycle, which converts formaldehyde (CH₂OH) into multi-carbon compounds for building cell biomass. The majority of Group I methanotrophs are grouped into the Methylococcaceae family. Group II MB are alphaproteobacteria, contain rings of particulate methane monooxygenase (pMMO)-harboring membranes at the cell periphery, generally accumulate C18 fatty acids, and use the serine cycle for converting formaldehyde into biomass. Methylocystis and Methylosinus species are typical representatives of Group II MB. Group III MB do not produce intracellular membranes (ICM), display a low growth rate, and assimilate carbon through the Calvin-Benson-Bassham (CBB) cycle. Group III MP are represented by methanotrophic Verrucomicrobia.

The biotechnological potential of MB has been of broad interest for decades, ranging from bioremediation to biocatalysis, such as the production of specialty chemicals, polymers, and food-grade chemicals (epoxides, poly-β-hydroxybutyrate, ectoine, and astaxanthin) or single cell protein. In the past, major efforts have taken place in the UK, Denmark, and USSR to develop industrial-scale processes for converting methane into single cell proteins (SCP) using Type I MB for high yield biomass production. For example, in the USSR, production fermentors (up to 750 m³ working volume), were used to produce about 36,000 tons of dry biomass a year at a cell density of 20 g/L. SCP from a methanotrophic consortium (BioProtein, Norferm Danmark A/S), is a commercially established product in Denmark with the isolated protein product being approved by the European Union for use as a feed for salmon, calves, and pigs.

Recent efforts in culturing novel methanotrophic species have resulted in isolation and characterization of a variety of novel methoanotrophs. For example, extremely thermophilic, psychrophilic, acidophilic, alkaliphilic, and halophilic methanotrophs have been isolated, thus expanding the physiological range of aerobic methanotrophy (Khmelenina et al., 1999; Kalyuzhnaya et al., 2001; 2008). These microbes provide a multitude of potential applications for biotechnology (Jiang et al., 2010; Trotsenko Y A, & N. Khmelenina. 2008). Alkaliphilic/tolerant and halophilic/tolerant methanotrophs related to the genus Methylomicrobium are becoming a highly recognized and desired system for bioprocess engineering, due to the high growth and methane oxidation rates, and tolerance of a wide range of environmental conditions.

Haloalcaliphilic methanotrophs grow extremely well in pure culture, and are resistant to a variety of water chemistries and contaminants as well as typical gaseous impurities found in natural gas. All of these parameters make strains 5GB1 and 20Z particularly well suited for developing a modular system to be reproduced at low-grade gas sources, making use of local available water resources (including freshwater, brackish, or marine water).

As the base case, the 20Z strain naturally makes up to 1.5% of dry cell weight as sucrose (But et al., 2013).

### Metabolic Networks

All known aerobic MB use methane monooxygenase (MMO) for the first oxidation step that converts methane into methanol and the cells further oxidize methanol to formaldehyde, formate, and then into carbon dioxide. Two isoenzymes of MMO are known: soluble methane monooxygenase (sMMO) and membrane bound (or articulate) methane monooxygenase (pMMO). pMMO has a higher affinity for methane compared to sMMO, and pMMO is the most efficient system for methane oxidation.

Group I MB also use a more efficient pathway for assimilation of the C1 unit into biomass than other MB. In these strains, formaldehyde is directly assimilated to form central metabolic intermediates by the assimilatory RuMP pathway. This pathway allows Group I MB to generate the highest cellular yields from methane.

Sucrose synthesis and accumulation are attributes of most photosynthetic eukaryotes and some species of photoautotrophic prokaryotes (Klan & Hagemann, 2011). The described biochemical pathway for sucrose biosynthesis involves the sucrose-phosphate synthase (Sps, UDP-glucose: D-fructose-6-phosphate 2-α-D-glucosyltransferase, EC 2.4.1.14) and sucrose-phosphate phosphatase (Spp, sucrose-6-phosphohydrolase, E.C. 3.1.3.24) (Bruneau et al., 1991; Page-Sharp et al., 1999; Lunn et al., 2000). Phylogenetic studies on Sps and Spp have shown that the cyanobacterial and plant enzymes are closely related (Lunn, 2002). Little has been known about enzymes involved in the sucrose biosynthetic pathway in non-phototrophic bacteria

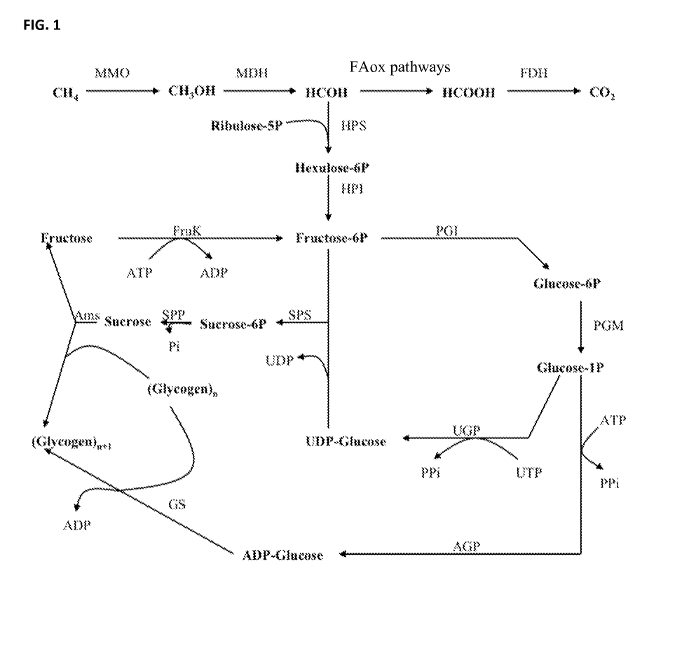

Cells of M. alcaliphilum 20Z produce sucrose in response to increased salinity of the growth media (Khmelenina et al., 1999). The inventors have identified all of the steps essential for conversion of methane into sucrose (FIG. 1). Four genes encoding the putative enzymes Sps, Spp, fructokinase (FruK) and amylosucrase (Ams) are clustered together. Corresponding proteins were purified and characterized (Table 1).

TABLE 1
Properties of the sucrose-phosphate phosphatase (Sps)
and amylosucrase (Ams) from M. alcatiphilum 20Z

| | Sps | Ams | |
|---|---|---|---|
| Subunit molecular | 31.4 | 76 | |
| mass, kDa | | | |
| Subunits structure | monomer, dimer, | monomer | |
| | tetramer, hexamer | | |
| pH optimum′ | 6.5 | 8.0 | |
| Temperature | 35 | 30 | |
| optimum (° C.) | | 8.1 (11.3)* | [Total] |
| Kₘ (mM) | 0.036 | 6 (11.2) | [Transglycosilation] |
| | | 11 (11)* | [Hydrolysis] |
| Vₘₐₓ (U/mg) | 18.9 | — | |
| k_{cat} (min⁻¹) | — | 8.7 (11.2)* | [Total] |
| | | 4.6 (7.5) | [Transglycosilation] |
| | | 4.1 (4.0)* | [Hydrolysis] |

| | | |
|---|---|---|
| Kᵢ (sucrose) | 1000 | — |
| (mM) | | |

*the reaction was performed in the presence of 0.1 mg/ml glycogen.

Theoretical parameters for sucrose production in methanotrophic bacteria are summarized as:

26CH₂O+2O₂=2C₁₂H₂₂O₁₁+4H₂O+2CO₂

### CCE-92%

Y[sucrose]=1.6 g (1.6 g sucrose per 1 g CH4 consumed)

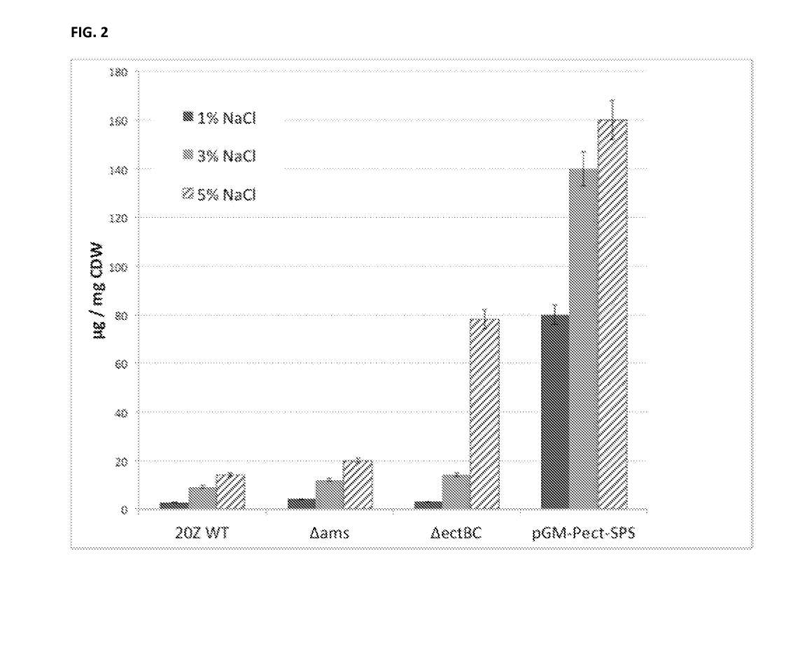

Input Parameters
1) Sucrose production balance from formaldehyde (from FIG. 2):

12CH₂O+UTP+2PPi+H₂O=C₁₂H₂₂O₁₁+5Pi+UDP (2)

PPi+H₂O=2Pi (2a)
(3) formaldehyde oxidation balance

CH₂O+2NAD+H₂O═CO₂+2NADH/H⁺
(4) Respiration balance:

2NADH/H⁺+O₂±5ADP+5Pi=2NAD+2H₂O+5ATP
(5) PPi-ase:

ATP+5Pi=3PPi+ADP

UDP+ATP=ADP+UTP (6)

Similar pathways were also identified in 12 (out of 19 tested) genomes of methanotrophic bacteria belonging to Gammaproteobacteria (Table 2). Thus, it is explicitly contemplated that gammaproteobacteria can also be genetically engineered in a manner similar to the Group I methanotrophic bacteria described herein.

TABLE 2
Sucrose-biosynthesis pathway distribution
among methanotrophic bacteria

| Methanotrophs | sps | spp | fruK | ams |
|---|---|---|---|---|
| Gammaproteobacteria | | | | |
| Methylomicrobium buryatense 5G | + | + | + | + |
| Methylobacter luteus | + | + | + | + |
| Methylocaldum szegediense | + | + | + | − |
| Methylobacter marinus | + | + | + | + |
| Methylobacter tundripaludum | + | + | + | + |
| Methyloglobulus morosus KoM1 | + | + | + | + |
| Methylohalobius crimeensis | + | − | + | − |
| Methylomicrobium album | + | + | + | + |
| Methylomicrobium alcaliphilum | + | + | + | + |
| Methylomonas methanica | + | + | + | + |
| Methylomonas sp LW13 | + | − | + | + |
| Methylosarcina fibrata | + | + | + | + |
| Methylomonas sp. MK1 | − | − | − | − |
| Methylomonas sp. 11b | − | − | − | − |
| Methylosarcina lacus LW14 | − | − | − | − |
| Methylobacter sp. 31-32 | − | − | − | − |
| Methylovulum miyakonense strain HT12 | − | − | − | − |
| Methylococcus capsulatus Bath | − | − | − | − |
| Methylococcus capsulatus Texas | − | − | − | − |
| Alphaproteobacterial methanotrophs | − | − | − | − |
| Verrucomicrobial methanotrophs | − | − | − | − |

While particular examples of genes for modification are described, the current disclosure also encompasses modifications to genes that hybridize with the specifically disclosed genes or are otherwise structurally and functionally related to the genes described. In this manner, for example, genes with functionally redundant counterparts can also be targeted to modify carbon flux in a desired MB. A gene or polynucleotide fragment “hybridizes” to another gene or polynucleotide fragment, such as a cDNA, genomic DNA, or RNA, when a single stranded form of the polynucleotide fragment anneals to the other polynucleotide fragment under the appropriate conditions of temperature and solution ionic strength. Hybridization and washing conditions are well known, and exemplified in, e.g., Sambrook, J., Fritsch, E. F. and Maniatis, T. Molecular Cloning: A Laboratory Manual, Second Ed., Cold Spring Harbor Laboratory Press, Cold Spring Harbor (1989), particularly Chapter 11 and Table 11.1 therein (incorporated by reference herein for its teachings regarding the same). The conditions of temperature and ionic strength determine the “stringency” of the hybridization. Stringency conditions can be adjusted to screen for moderately similar fragments (such as homologous sequences from distantly related organisms) to highly similar fragments (such as genes that duplicate functional enzymes from closely related organisms). Post-hybridization washes determine stringency conditions. One set of hybridization conditions to demonstrate that sequences hybridize uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Stringent conditions use higher temperatures in which the washes are identical to those above except for the temperature of the final two 30 min washes in 0.2×SSC, 0.5% SDS is increased to 60° C. Highly stringent conditions use two final washes in 0.1 SSC, 0.1% SDS at 65° C. Those of ordinary skill in the art will recognize that these temperature and wash solution salt concentrations may be adjusted as necessary according to factors such as the length of the hybridizing sequences. Other approaches based on hybridization include, for example, the expression of antisense sequences to target a desired gene.

Proteins and genes that share a % identity with the proteins and genes explicitly disclosed herein are also within the scope of the present disclosure. The % identity is at least 85%, at least 86%, at least 87% at least 88%, at least 89%, at least 90%, at least 91%, at least 92%, at least 93%, at least 94%, at least 95%, at least 96%, at least 97%, at least 98% or at least 99%. As is known in the art, “% identity” refers to a relationship between two or more protein sequences or two or more polynucleotide sequences, as determined by comparing the sequences. In the art, “identity” also means the degree of sequence relatedness between proteins or polynucleotides as determined by the match between strings of such sequences. “Identity” (often referred to as “similarity”) can be readily calculated by known methods, including (but not limited to) those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992), each incorporated by reference herein for its teachings regarding the same. Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp (1989) CABIOS. 5:151-153, incorporated by reference herein for its teaching regarding the same) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410, 1990, incorporated by reference herein for its teaching regarding the same); DNASTAR (DNASTAR, Inc., Madison, Wis.); and the FASTA program incorporating the Smith-Waterman algorithm (Pearson, Comput. Methods Genome Res., (Proc. Int. Symp.) (1994), Meeting Date 1992, 111-20. Editor(s): Suhai, Sandor. Publisher: Plenum, New York, N.Y. incorporated by reference herein for its teaching regarding the same). Within the context of this disclosure it will be understood that where sequence analysis software is used for analysis, the results of the analysis are based on the “default values” of the program referenced. As used herein “default values” will mean any set of values or parameters which originally load with the software when first initialized.

### Modification of Methanotroph Bacteria

Provided herein are modified Group I methanotrophic bacteria that can convert methane into commercially valuable products, for example Methylomicrobium alcaliphilum 20Z that can convert methane and methanol into sucrose.

Appropriate Group I strains for modification include without limitation, 5GB1, 20Z, Methylobacter bovis, Methylomonas sp. LW13 (LW13), Methylomonas MK1 (“MK1”) and Methylomonas sp.11b (“sp.11b”). In some embodiments, methane refers to CH₄. In some embodiments, the methods and compositions described herein relate to the conversion of CH₄.

CH₄ can be obtained from a variety of sources, including, by way of non-limiting example, natural gas, fracking, landfill emissions, livestock facilities, Fischer-Tropsch processes, coal seam gas, and the fermentation of wastewater sludge, manure, and/or solid waste. It is specifically contemplated herein that the CH₄ used in the methods described herein can be gaseous methane from one or more of the foregoing sources.

In particular embodiments, the modifications result in methanobacteria (MB) with e.g., up-regulated sucrose content, up-regulated methane flux, up-regulated carbon conversion efficiency, up-regulated carbon conversion into cellular sugars, up-regulated sucrose synthesis, and down-regulated carbon conversion into glycogen and/or down-regulated sucrose degradation pathways.

Such modifications can be achieved, for example, by up-regulating activity of pathways that convert fructose-6-phosphate and UDP-glucose into sucrose, down-regulating/deletion of ectoine biosynthesis pathway (ectBC genes). The described modifications can also be achieved by up-regulating methane monooxygenase, hexulose phosphate synthase and phospho-3-hexuloisomerase, sucrose-phosphate synthase and downregulation/deletion of amylase, gluconate-6-phosphate dehydrogenase, glucose-1-phosphate adenyltransferase, ADP-glucose pyrophosphorylase glycogen synthase, glycogen branching enzyme.

As used herein, “up-regulation” or “up-regulated” means increasing an activity within a bacterial cell. The activity can be the actions of one or more metabolic pathways or portions of metabolic pathways within a bacterial cell. An up-regulation of one activity can be caused by the down-regulation of another. Alternatively, an up-regulation of an activity can occur through increased activity of an intracellular protein, increased potency of an intracellular protein or increased expression of an intracellular protein. The protein with increased activity, potency or expression can be encoded by genes disclosed herein.

To cause an up-regulation through increased expression of a protein, the copy number of a gene or genes encoding the protein can be increased. Alternatively, a strong and/or inducible promoter can be used to direct the expression of the gene, the gene being expressed either as a transient expression vehicle or homologously or heterologously incorporated into the bacterial genome. In another embodiment, the promoter, regulatory region and/or the ribosome binding site upstream of the gene can be altered to achieve the over-expression. The expression can also be enhanced by increasing the relative half-life of the messenger or other forms of RNA. Any one or a combination of these approaches can be used to effect upregulation of a desired target protein as necessary for the methods and compositions described herein.

As used herein, “down-regulation” or “down-regulated” means any action at the metabolic pathway, protein or gene level that results in: a decrease in the activity of a metabolic pathway or a portion thereof; a decrease in activity of a protein; elimination of a protein's activity, translation of an incomplete protein sequence; incorrect folding of protein; reduced transcription of a gene; incomplete transcription of a gene, interference with an encoded RNA transcript, or any other activity resulting in reduced activity of a pathway, protein or gene. An increase in the expression of a pathway inhibitory protein or signaling molecule can also result in pathway downregulation.

A gene can be down-regulated for example by insertion of a foreign set of base pairs in a coding region, deletion of any portion of the gene, or by the presence of antisense sequences that interfere with transcription or translation of the gene. In another embodiment, down-regulation includes elimination of a gene's expression (i.e. gene knockout). As used herein, the symbol “A” denotes a mutation in the specified coding sequence and/or promoter wherein at least a portion (up to and including all) of the coding sequence and/or promoter has been disrupted by a deletion, mutation, or insertion. In another embodiment, the disruption can occur by optionally inserting a nucleotide or polynucleotide molecule into the native gene sequence whereby the expression of the mutated gene is down-regulated (either partially or completely). Any one or a combination of these approaches can be used to effect downregulation of a desired target protein as necessary for the methods and compositions described herein.

“Up-regulation” and “down-regulation” can be measured against a control condition including, without limitation, relative to the activity of an unmodified bacterial strain of the same species.

Embodiments disclosed herein include modified Group I MB that achieve a sucrose content of at least 0.1%, at least 0.2%, at least 0.3%, at least 0.4%, at least 0.5, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49% or at least 50%.

In another embodiment, the modified Group I MB comprises a carbon conversion efficiency of at least 45%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even 100%. In additional embodiments, the modified Group I MB utilizes a variant of the RuMP pathway which involves glycolysis. In additional embodiments, the modified Group I MB are modified 5GB1, modified 20Z, modified MK1 or modified sp.11b. In a particular embodiment the modified strain is 20ZER-S1 with a sucrose content of at least 7% and a carbon conversion efficiency of at least 60%.

In other embodiments, the sucrose content or sucrose production of a modified or engineered methanotrophic bacterium is at least 20% higher than the sucrose content or production of the corresponding wild-type methanotrophic bacterium under the same growth conditions. For example, the sucrose content or production of the engineered methanotrophic bacterium is at least 25%, at least 30%, at least 40%, at least 50%, at least 60%, at least 70%, at least 80%, at least 90%, at least 95%, at least 98%, at least 99%, at least 1-fold, at least 2-fold, at least 5-fold, at least 10-fold, at least 100-fold or higher than the sucrose content or production of the corresponding wild-type methanotrophic bacterium under the same growth conditions.

### Modification of Sucrose Biosynthesis Pathways

A central goal of the strain modification approach is to increase flux of carbon into sucrose biosynthesis pathways. Analysis of corresponding metabolic pathways has resulted in identification of several metabolic steps for which elimination or activation leads to increased flux of carbon into the sucrose biosynthesis pathway. Identified targets for genetic modifications for improvements for sucrose productivity can include: increased gene copy numbers for sucrose-phosphate synthase (sps) to increase sucrose production; deletion of ectoine biosynthesis genes (ectBC) to reduce ectoine production and stimulate sucrose accumulation; deletion of amylase (ams) to reduce sucrose degradation.

Another modification can include deletion of specific genes, including e.g., gluconate-6-phosphate dehydrogenase (gnd) in order to increase carbon conversion by down-regulating the cyclic oxidation of formaldehyde through the pentose-phosphate pathway and redirect carbon into sucrose biosynthesis.

Based on the foregoing, and without wishing to be bound by theory, a summary of modifications includes, without limitation:
1. Up-regulating sucrose production by eliminating or down-regulating the ectoine biosynthesis pathway;
2. Up-regulating sucrose production by eliminating or down-regulating sucrose-cleavage (deletion of ams gene);
3. Up-regulating sucrose production by eliminating or down-regulating glycogen biosynthesis

While particular examples of genes for modification are described, the current disclosure also encompasses modifications to genes that hybridize with the specifically disclosed genes. A gene or polynucleotide fragment “hybridizes” to another gene or polynucleotide fragment, such as a cDNA, genomic DNA, or RNA, when a single stranded form of the polynucleotide fragment anneals to the other polynucleotide fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Ed., Cold Spring Harbor Laboratory Press, Cold Spring Harbor (1989), particularly Chapter 11 and Table 11.1 therein (incorporated by reference herein for its teachings regarding the same). The conditions of temperature and ionic strength determine the “stringency” of the hybridization. Stringency conditions can be adjusted to screen for moderately similar fragments (such as homologous sequences from distantly related organisms) to highly similar fragments (such as genes that duplicate functional enzymes from closely related organisms). Post-hybridization washes determine stringency conditions. One set of hybridization conditions to demonstrate that sequences hybridize uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Stringent conditions use higher temperatures in which the washes are identical to those above except for the temperature of the final two 30 min washes in 0.2×SSC, 0.5% SDS is increased to 60° C. Highly stringent conditions use two final washes in 0.1SSC, 0.1% SDS at 65° C. Those of ordinary skill in the art will recognize that these temperature and wash solution salt concentrations may be adjusted as necessary according to factors such as the length of the hybridizing sequences.

Chromosomal DNA from M. alcaliphilum cells was prepared as previously described (Kalyuzhnaya et al., 2008). Putative sps (GenBank CCE22309.1), spp (CCE22310.1) and ams (CCE22312.1) genes were amplified by PCR. The PCR-products were purified on a Wizard column (Promega, USA), incubated with the endonucleases and ligated in the expression vector pET30(a)+ between NdeI and HindIII sites (sps and ams genes) or in the pET28b vector between NcoI and HindIII sites (spp gene). Cells of E. coli Rosetta (DE3) were transformed by the resulting vector, grown overnight at 37° C. in 20 mL of LB medium, transferred into fresh LB medium containing 50 μg/ml kanamycin and 25 μg/ml chloramphenicol, and cultivated until OD₆₀₀ 0.6-0.7. Protein expression was induced by isopropyl β-D-1-thiogalactopyranoside (IPTG) at a final concentration of 0.5 mM. After overnight incubation at 17° C. the cells were harvested by centrifugation at 6000 g for 20 min (4° C.). The His₆-tagged proteins were purified by affinity chromatography on a Ni²⁺-NTA column as earlier described (But et al., 2012), and their purity was analyzed by 12% SDS-PAGE (Laemmli, 1970).

To obtain a deletion in the sps gene, coding for SPS, the DNA fragment containing the ORF coding for SPS and the region upstream of the ectABC operon (287 bp) containing the promoter sequence (Mustakhimov et al., 2010) were amplified from chromosomal DNA by using the primers sps-CF (containing a SacI restriction site and the ribosome-binding sequence), sps-CR (containing a PciI restriction site) and primers Pect-F and Pect-R (containing recognition sites for EcoRI and SacI endonucleases, respectively), treated with an appropriate endonuclease and ligated to the vector pCM160 digested by the same enzymes. The DNA fragment containing the sps gene was treated by SacI and PciI endonuclease and ligated to vector pCM160 digested by the same enzymes. The Pect promoter was cloned into the vector pCM-sps between the EcoRI and SacI sites. The kanamycin cassette of the plasmid pCMPect-sps was exchanged to a gentamycin cassette, which was cut from the plasmid p34S-Gm by the PstI endonuclease. The resulting vector pGmPect-sps was transferred into E. coli S-17-1 and further to the mutant Asps by conjugation as described above. The mutant cells harboring the plasmid were selected on solid medium 2P with 3% NaCl containing kanamycin (100 μg/ml) and gentamycin (15 μg/ml).

Proteins and genes that share a % identity with the proteins and genes explicitly disclosed herein are also within the scope of the present disclosure. The % identity is at least 85%, at least 86%, at least 87% at least 88%, at least 89%, at least 90%, at least 91%, at least 92%, at least 93%, at least 94%, at least 95%, at least 96%, at least 97%, at least 98% or at least 99%. As is known in the art, “% identity” refers to a relationship between two or more protein sequences or two or more polynucleotide sequences, as determined by comparing the sequences. In the art, “identity” also means the degree of sequence relatedness between proteins or polynucleotides as determined by the match between strings of such sequences. “Identity” (often referred to as “similarity”) can be readily calculated by known methods, including (but not limited to) those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992), each incorporated by reference herein for its teachings regarding the same. Preferred methods to determine identity are designed to give the best match between the sequences tested. Methods to determine identity and similarity are codified in publicly available computer programs. Sequence alignments and percent identity calculations can be performed using the Megalign™ program of the LASERGENE™ bioinformatics computing suite (DNASTAR™, Inc., Madison, Wis.). Multiple alignment of the sequences can also be performed using the Clustal method of alignment (Higgins and Sharp (1989) CABIOS. 5:151-153, incorporated by reference herein for its teaching regarding the same) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410, 1990, incorporated by reference herein for its teaching regarding the same); DNASTAR™ (DNASTAR™, Inc., Madison, Wis.); and the FASTA program incorporating the Smith-Waterman algorithm (Pearson, Comput. Methods Genome Res., (Proc. Int. Symp.) (1994), Meeting Date 1992, 111-20. Editor(s): Suhai, Sandor. Publisher: Plenum, New York, N.Y. incorporated by reference herein for its teaching regarding the same). Within the context of this disclosure it will be understood that where sequence analysis software is used for analysis, the results of the analysis are based on the “default values” of the program referenced. As used herein “default values” will mean any set of values or parameters which originally load with the software when first initialized.

### Dry Fermentation

The methods described herein relate, in part, to the use of methanotrophic bacterial compositions to convert methane to sucrose, which can be applied to a process for removing methane from a gaseous waste stream. The engineered methanotrophic bacteria described herein are stimulated to convert methane to the biotechnologically useful compound sucrose under conditions where production of an osmoprotecant (e.g., sucrose) is desirable. Such conditions can include dry or arid conditions, or high salt conditions. Thus, the aerobic fermentation of methane to sucrose, rather than to another metabolic process, is increased under dry conditions and is referred to herein as “dry fermentation.”

Dry fermentation is typically performed using methanotrophic bacteria attached to a solid support, such as a filter, over which the gaseous substrate comprising methane is passed. For example, the filter can be placed in a pipe or a chamber designed to increase surface area of the filter, through which methane is passed. Multiple filter units (e.g., 2, 3, 4, 5, 6, 7, 8, 9, 10, 20, 50, 100, or more) can be used in series to ensure that most, if not all, of the methane is extracted from the gaseous substrate or waste stream. In order to remove sucrose, the filters are washed with water to extract the sucrose form the filter during a “sucrose removal cycle.” Such cycles can vary in frequency (e.g., hourly, daily, weekly, etc) or length of time (e.g., 5 min, 10 min, 20 min, 30 min, 60 min, 2 h, 6 h, etc). The sucrose can be collected in a collecting device for further treatment (e.g., removal of amino acids or cell lysis fragments), as desired, prior to use in other biotechnologies. Following removal of the sucrose, the filters are essentially reset to the original dry conditions and dry fermentation is continued. It is contemplated herein that nutrients or substrates, such as nitrates, sulfates or phosphates, can be added following a sucrose removal cycle to support the bacteria during the next round of dry fermentation. The set-up can be monitored for methane escape through the filters by using a biogas sensor, which is available through a variety of commercial sources including e.g., BluSens™. It is also contemplated herein that the gaseous substrate that has passed over the filters and is depleted in methane can be passed through another chamber comprising additional filter units or alternatively, can be recycled back through the original filter units to ensure complete or more efficient methane extraction by the biofilters.

Any variety of filter can be used provided that the filter composition does not interfere with the viability and/or growth of the methanotrophic bacteria and further is stable under the optimal growth conditions and during exposure to the gaseous substrate. In one embodiment, the filter is a mesh. In such embodiments, the mesh comprises a size that is large enough to permit passage of the gaseous substrate but not too large that the bacteria will pass through. In some embodiments, the mesh comprises a pore size of 0.2-0.5 μm. In other embodiments, the filter comprises a pore size of 0.2-0.3 μm, 0.2-0.4 μm, 0.4-0.5 μm, or 0.2-0.3 μm. In one embodiment, the filter is a 0.22 μm filter. In another embodiment, the filter is a 0.45 μm filter.

The filter units can be replaced once they are no longer functional as assessed by measuring the amount of escaped methane, or when they dry out to a reduced production of metabolic water. While one feature of the technology described herein is the discovery that engineered methanobacteria can remain viable and active for methane fermentation for extended periods of months or more, to the extent that methane conversion or sucrose production may decrease with filter age or extended re-use, the filters can be replaced every month, every two months, every three months, every four months, every five months, every 6 months or so as necessary, e.g., to maintain effective removal of methane from a waste stream or to maintain levels of sucrose production at a desired rate.

The methanotrophic bacteria can be seeded on the filter at any preferred cell density provided that the number of cells does not impede flow of the gaseous substrate through the filter. Typically, the cell density will range from 0.25-5 g/cm², 0.25-4 g/cm², 0.25-3 g/cm², 0.25-2 g/cm², 0.25-2 g/cm², 0.25-0.5 g/cm², 0.5-5 g/cm², 1-5 g/cm², 2-5 g/cm², 3-5 g/cm², 4-5 g/cm², 1-3 g/cm², or 2-4 g/cm².

As will be recognized by one of skill in the art, a variety of growth conditions (e.g., temperature) can be modified to determine and apply an optimal set of conditions for the production of sucrose from methane.

In some embodiments, the growth conditions comprise a temperature range between 15-37° C., between 15-36° C., between 15-35° C., between 15-34° C., between 15-33° C., between 15-32° C., between 15-31° C., between 15-30° C., between 15-25° C., between 15-20° C., between 20-37° C., between 25-37° C., between 30-37° C., between 31-37° C., between 32-37° C., between 33-37° C., between 34-37° C., between 35-37° C., between 36-37° C., between 25-30° C., between 20-30° C., between 25-32° C., or any range therebetween.

In general, the gaseous substrate comprising methane is provided at atmospheric pressure, however it is also contemplated herein that pressures higher than atmospheric pressure can be used if so desired (e.g., 14.7-60 psi, 15-60 psi, 20-60 psi, 30-60 psi, 40-60 psi, 50-60 psi, 14.7-50 psi, 14.7-40 psi, 14.7-30 psi, 14.7-20 psi, or 20-40 psi).

In some embodiments, the gaseous stream is diluted or concentrated to obtain a desired concentration of methane. For example, the range of methane concentration in a gaseous stream can be from 0.1-50%, from 0.5-50%, from 1-50%, from 5-50%, from 10-50%, from 20-50%, from 25-50%, from 30-50%, from 40-50%, from 0.1-40%, from 0.1-30%, from 0.1-25%, from 0.1-20%, from 0.1-10%, from 0.1-5% from 0.1-1%, from 0.1-0.5% or from 25-40%.

Typically, oxygen from ambient air will be sufficient to sustain aerobic conditions necessary for dry fermentation, however it is also contemplated herein that oxygen can be added to the gaseous mixture to generate a set of desired aerobic conditions.

In some embodiments, the humidity is maintained at between 30%-98%, between 30-95%, between 30%-90%, between 30%-80%, between 30-75%, between 30-70%, between 30-60%, between 30-50%, between 30-40%, between 40-80%, between 50-70%, between 50-80%, between 50-90%, between 70-80%, between 70-90%, between 70-95% or between 70-98%.

### Industrial Production

A variety of culture methodologies may be applied to the modified strains described herein. For example, large-scale production of a specific product made possible by the modified strains described herein may be accomplished by both batch and/or continuous culture methodologies.

A classical batch culturing method is a closed system where the composition of the media is set at the beginning of the culture and not subject to external alterations during the culturing process. Thus, at the beginning of the culturing process the medium is inoculated with the desired strain and growth or metabolic activity is permitted to occur adding nothing to the system. Typically, however, a “batch” culture is batch with respect to the addition of carbon source and attempts are often made at controlling factors such as pH and oxygen concentration. In batch systems the composition of the system changes constantly up to the time the culture is terminated. Within batch cultures, strain cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable and comprise a typical batch system with the exception that the substrate is added in increments as the culture progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227 (1992) each of which is incorporated by reference herein for its teachings regarding the same.

Continuous cultures can also be used. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture can be practiced with immobilized cells where carbon and nutrients are continuously added and valuable products, by-products, and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method maintains a limiting nutrient such as the carbon source or nitrogen level at a fixed rate and allows all other parameters to moderate. In other systems, a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock Biology of Microorganisms, 8th edition, Prentice Hall, UpperSaddle River, N.J. (1997) which is incorporated by reference herein for its teachings regarding the same.

Extractive fermentation with immobilized cells can be used. Possibilities for product removal can include hollow fiber culture (Aziz et al., 1995 in Environ. Sci. Technol. 29: 2574-2583); membrane-aerated biofilm reactor (described by Rishell et al. 2004 in Biotechnol Prog: 1082-90). For extraction a biotechnological process called “bacterial milking” can be applied (Sauer T. and Galinski E. A. 1998 in Biotechnol Bioeng. 57: 306-313) can also be applied.

Regarding Methylomicrobium buryatense and alcaliphilum strains particularly, the strains can be grown in a simple mineral medium (NMS) supplemented with salt and carbonate buffer. In batch culture, optimal growth occurred at pH 8.5-9.5 and with 0.75-3% NaCl. Under these conditions, a doubling time of 3-6 hr was achieved. Total sucrose content ranged from 0.1-2%. Cells grown at high salinity contain increased levels of sucrose compared to cells grown at low salt (FIG. 2).

TABLE 3
Growth Parameters

| Parameters | Comment | |
|---|---|---|
| NaCl range (optimum) | 0.1-9% | (0.75%) |
| T range (optimum/resistance) | 4-47° C. | (30° C./80° C.) |
| pH range (optimum) | 6-11 | (8.0-9.0) |

| | |
|---|---|
| Sucrose content | 1-2% of cell dry weight |

The modified MB disclosed herein can generate 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3.6 g sucrose/L fermentation broth/hr). In particular embodiments, each of these numerical values is a minimum value.

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 4^{th} ed., J. Wiley & Sons (New York, N.Y. 2012); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 4th ed., Cold Spring Harbor Laboratory Press (Cold Spring Harbor, N.Y. 2012); provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. “About” can be from about +/−20% to +/−1%. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

As will be understood by one of ordinary skill in the art, each embodiment disclosed herein can comprise, consist essentially of or consist of its particular stated element, step, ingredient or component.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{th} Edition or a dictionary known to those of ordinary skill in the art, such as the Oxford Dictionary of Biochemistry and Molecular Biology (Ed. Anthony Smith, Oxford University Press, Oxford, 2004).

### EXAMPLES

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Example 1

### Mutants

Construction of mutants was performed as previously described (Ojala et al., 2010). The following cloning vectors were used: pCM184, as a suicide vector (Marx& Lidstrom, 2004); pRK2013 (Ditta et al., 1985) as a helper plasmid, pCR2.1 (Invitrogen™) for cloning of PCR products. E. coli strains JM109 (34), S17-1 (Yanish-Perron et al., 1985) and Top 10 (Invitrogen™) were routinely cultivated at 37° C. in Luria-Bertani (LB) medium (Sambrook et al., 1989). The following antibiotic concentrations were used: Tet, 12.5; Kan 100 μg ml⁻¹; Amp 100 μg ml⁻¹, Rif, 100 μg ml⁻¹.

Data from the Methylomicrobium spp genome projects were used for designing primers flanking upstream and downstream regions of targeted genes. Representative primers include those listed in Table 4.

TABLE 4
Representative Primers used for amplification of flanking regions

| | | | |
|---|---|---|---|
| | Primer | | |
| Gene ID | name | Sequence | Description |

Methylomicrobium alcaliphilum 20Z

| | | | |
|---|---|---|---|
| Ectoine | SB-1 | TTAGATCTCGGCCAAAATTGGCGATGAGTTGGT | ectBC-up-F |
| biosynthesis | SB-2 | TACCATGGACGATGACGGCAGCCGGCTTATCGAC | ectBC-up-R |
| genes | SB-3 | TTCCGCGGGCACACTCTATAACCTGGATCAGCATG | ectBC-dw-F |
| ectBC | SB-4 | TAGAGCTCGCCTCATCCGCCTTGGTCAGTAC | ectBC-dw-R |
| Amylosucrase, | SB-5 | GAGACGTCGCTCAACCGACTCATTGACG | ams-up-F |
| alpha-amylase, | SB-6 | GTCAGCTGGGATGAATCGGCCTCATTCG | ams-up-R |
| ams | SB-7 | GAGGGCCCGTGGAATAGTAGCGCTAAAC | amp-dw-f |
| MALCv4_0617 | SB-8 | GAGAGCTCGATCAAAATCCGGCGTCGGGGGTCG | amp-dw-r |
| glgABC1- | MK-7 | GAGACGTCGCTCAACCGACTCATTGACG | glcA2-dw-AatII |
| glgABC2 | MK-8 | GTCAGCTGGGATGAATCGGCCTCATTCG | glgA2-dw-PvuII |
| MEALZv4_4028360_4048136 | MK-9 | GAGGGCCCGTGGAATAGTAGCGCTAAAC | glgA1-up-ApaI |
| | MK-10 | GAGAGCTCGATCAAAATCCGGCGTCGGGGGTCG | glgA1-up-Sac |
| | MK-11 | GAGGGGCCCGTGGAATAGTAGTGCTAAACAAT | glgA1- |
| | | | dw/F/ApaI |
| | MK-12 | GAGGAGCTCCGTCAAAGGACGCCGTGAGCCCAG | glgA1- |
| | | | dw/R/SacI |
| | MK-13 | GAGGACGTCCACAGCGGCTTTGACTGGATCG | glgA1- |
| | | | up/F/AatII |
| | MK-14 | GAGGGTACCGAGTTTACCGAGGTGGATTTCGCC | glgA1- |
| | | | up/R/KpnI |
| | MK-15 | GACCATGGCACAACGGCATATTGGATTGC | glgB1-up/NcoI |
| | MK-16 | GAGAATTCGCTGTCGGCATCTTTGATC | glgB1-up/EcoRI |

Upstream and downstream fragments were PCR amplified, cloned into pCR2.1, and then subcloned into pCM184. Each construct was verified by sequencing, Resulting vectors were introduced into a donor strain E. coli S17-1 via standard transformation procedure (Sambrook et al., 1989). The donor strain grown on LB-agar medium supplemented with appropriate antibiotic and the recipient Methylomicrobium strain grown on NMS-agar medium were mixed in a donor:recipient ratio of 1:2, and plated on the optimized mating medium (Ojala et al., 2010). Plates were incubated at 30° C. under a methane:air atmosphere (25:75) for 48 h, and cells were transferred from a mating medium onto selective plates. Rifamycin, high pH and 3% salinity were applied for counter-selection against the donor cells. The kanamycin resistant (Kan′) recombinants were selected and re-plated onto new plates. The identity of the double-crossover mutants was verified by diagnostic PCR with primers specific to the insertion sites. Constructed strains include those listed in Table 5.

TABLE 5
Constructed Strains

| Strains | Description | Parental strain |
|---|---|---|
| M. alcaliphilum 20ZR | Rif^{R} derivative | 20Z wild type |
| pBS2 | Δams::kan | 20ZR |
| pBS3 | ΔglgABC1manBQamyC::kan | 20ZR |
| pBS5 | ΔglgAB2amyAC | 20ZR |
| pBS6 | ΔglgABC1-glgABC2 | 20ZR |
| pBS7 | ΔectBC | 20ZR |
| pBS8 | 20Z Δsps::Pect-Sps | 20Z |

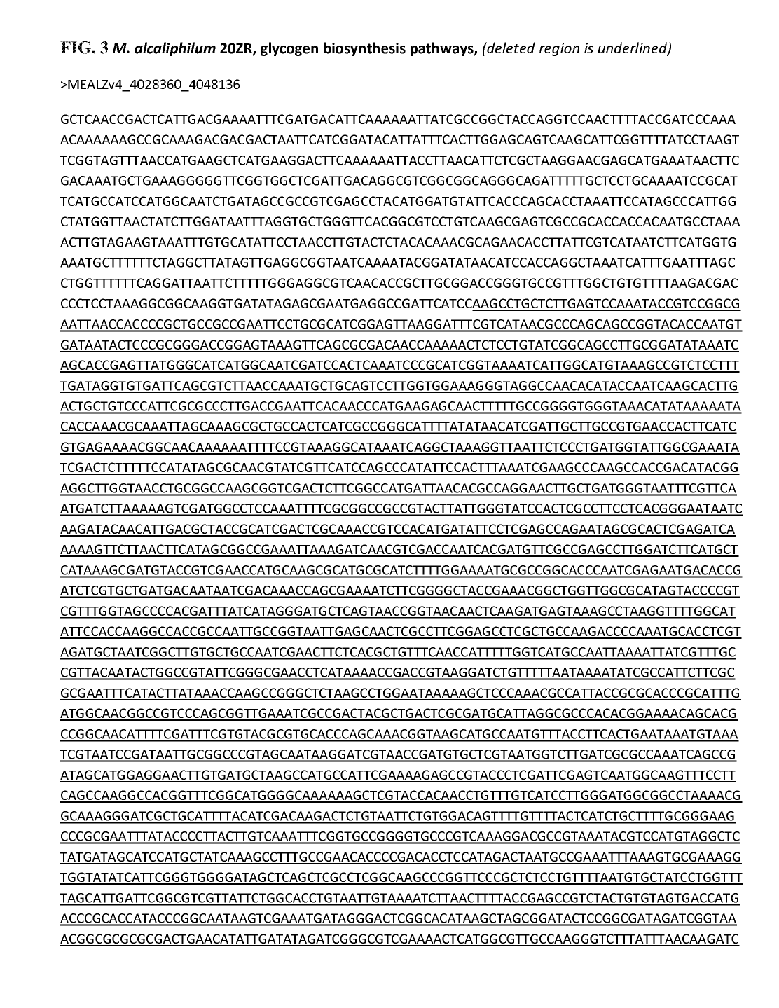

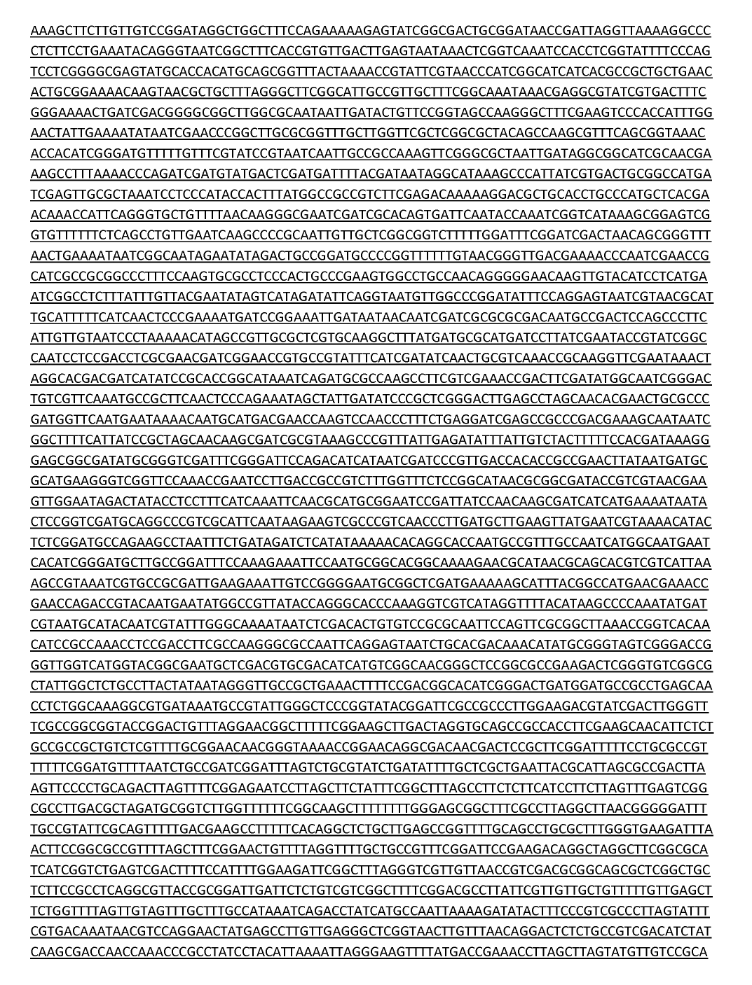

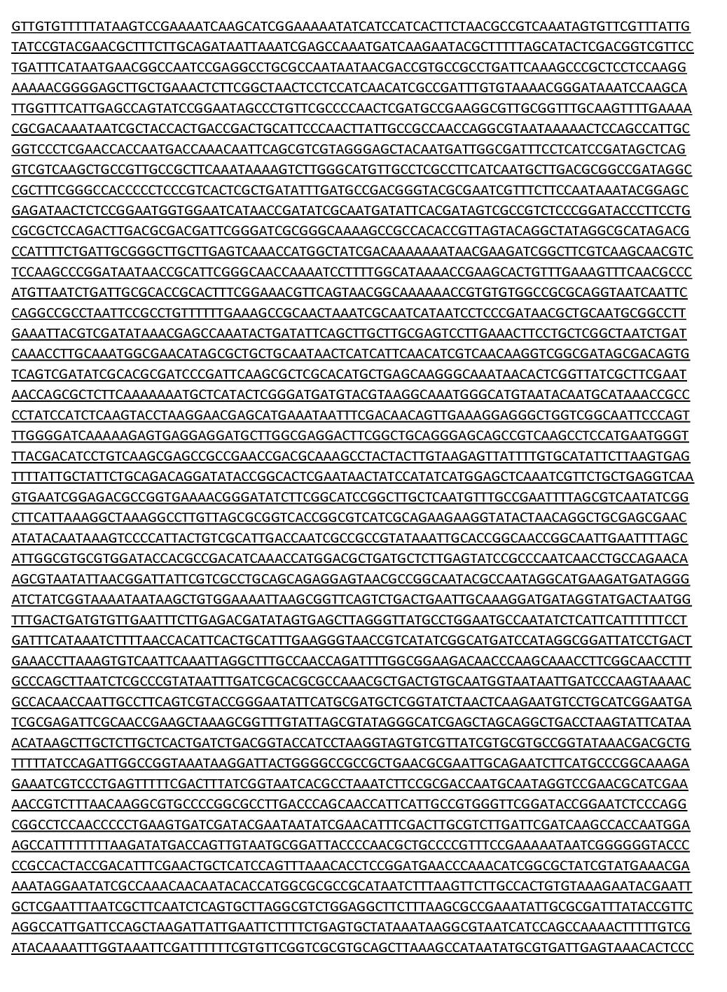

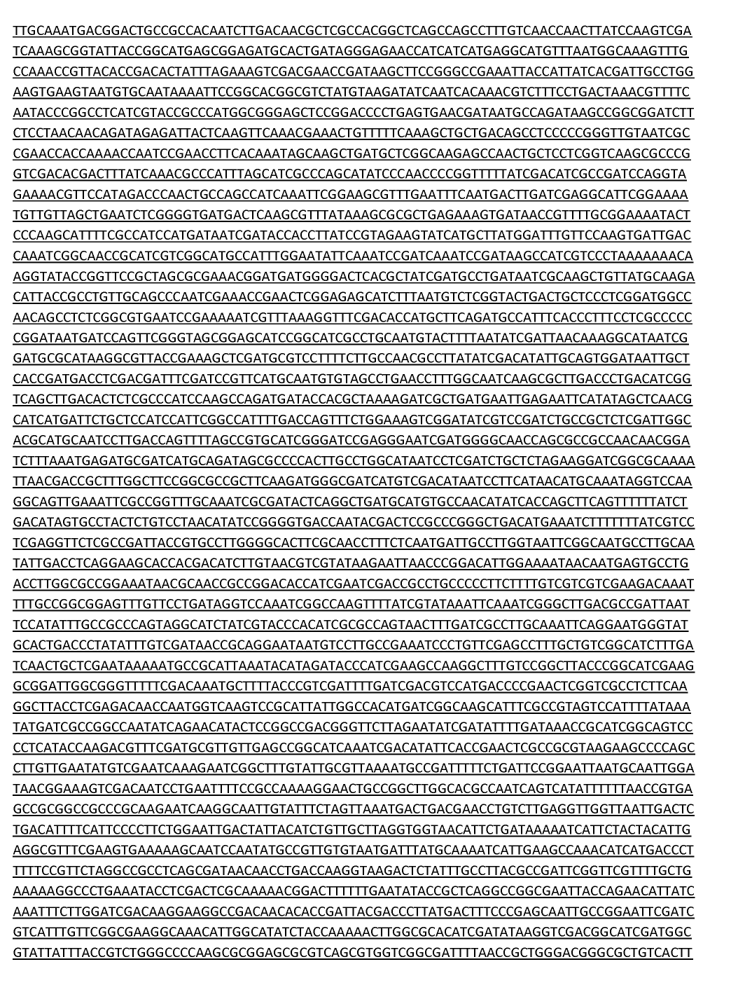

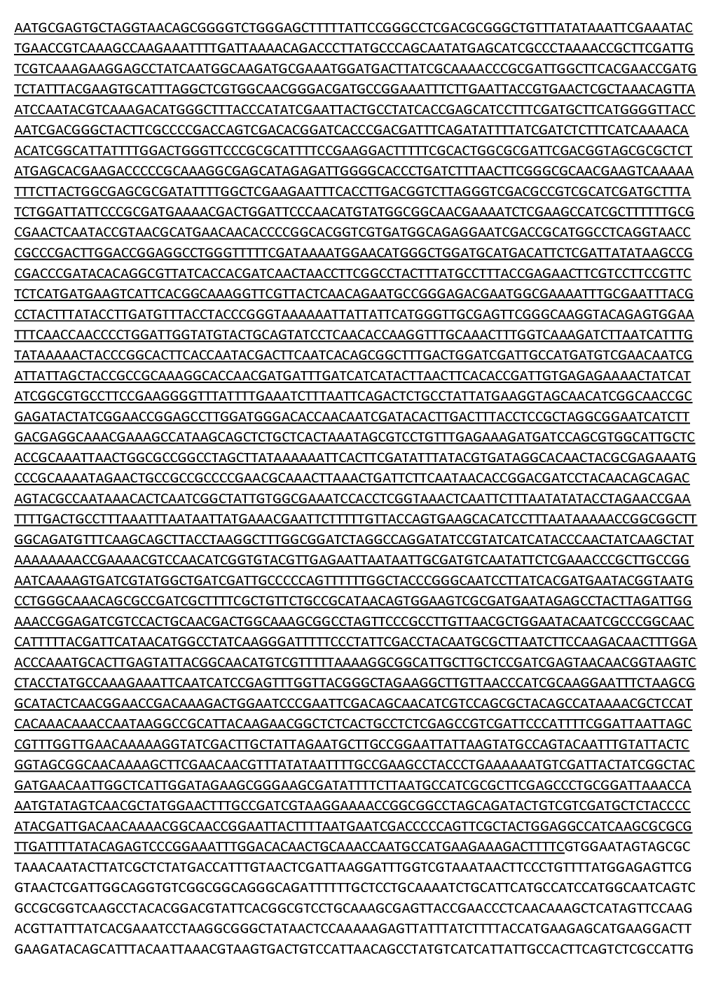

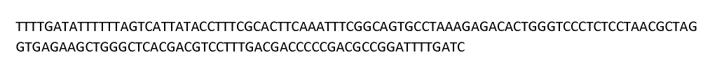

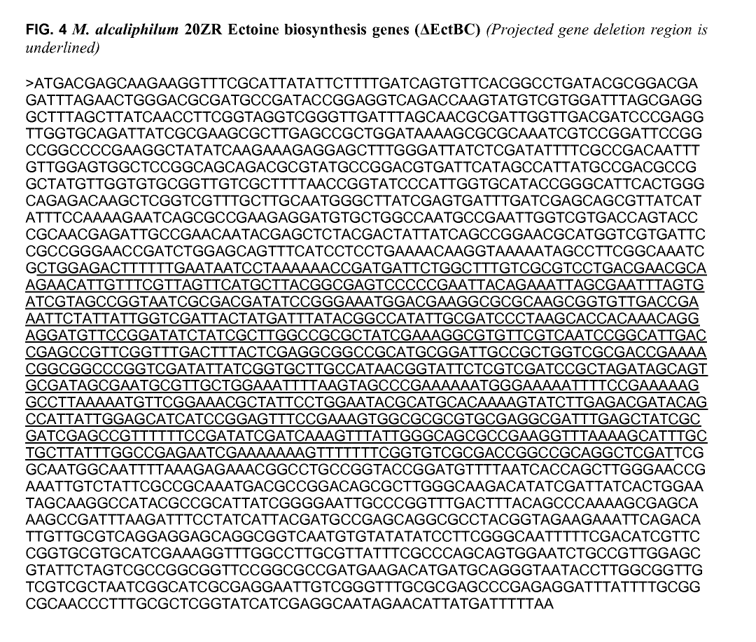

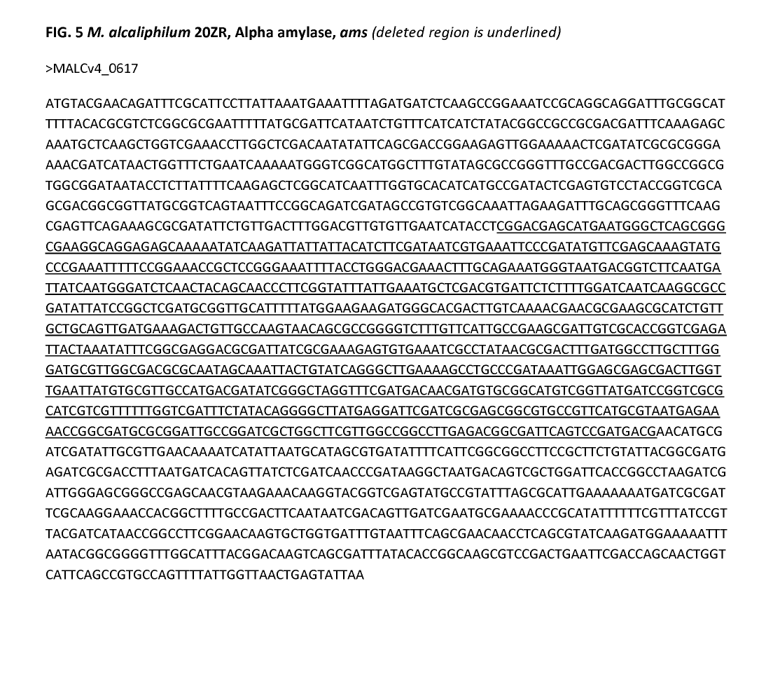

Characterization of sucrose accumulation in Methylomicrobium alcaliphilum 20Z wild type and mutant strains demonstrated that down-regulation of genes involved in ectoine biosynthesis or sucrose utilization results in increase of intracellular sucrose as shown in FIGS. 2, 3, and 4.

Each modification disclosed herein results in a modified Group I MB that has at least one of the following characteristics: sucrose content of 1%-25% wherein sucrose content is measured by the anthrone reagent alter solvent (methanol, methanol-chloroform) extraction; carbon conversion efficiency of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90% or more, wherein carbon conversion efficiency is percentage of total carbon incorporated into cell material and is calculated using the following equation:

CCE=([C mol in biomass]/[C mol of substrate consumed])*100; and/or

ability to generate at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5 or at least 3.6 g sucrose/L fermentation broth/hr of substrate.

As will be understood by one of ordinary skill in the art, each embodiment disclosed herein can comprise, consist essentially of or consist of its particular stated element, step, ingredient or

### REFERENCES

1. Anthony, C. 1982. The biochemistry of methylotrophs. London; New York: Academic Press 431p.
2. Aydin M., Verhulst, K. R., Saltzman, E. S., Battle, M. O., Montzka, S. A., Blake, D. R., Tang Q., & Prather, M. P. 2012. Recent decreases in fossil-fuel emissions of ethane and methane derived from firn air. Nature, 476: 198-201
3. Bruneau, J. M., Worrell, A. C., Cambou, B., Lando, D., Voelker, T. A., 1991. Sucrose phosphate synthase, a key enzyme for sucrose biosynthesis in plants. Plant Physiol. 96, 473-478.
4. But S. Iu., Khmelina V. N., Mustakhimova Trotsenko Iu. A. 2013. Production and characterization of Methylomicrobium alcaliphilum 20Z knockout mutants, which has sucrose and ectoin synthesis defective genes. Mikrobiologiia. 82(2): 251-3
5. Cuéllar A. D., & M. E Webber. 2008. Cow power: the energy and emissions benefits of converting manure to biogas. Environ. Res. Lett. 3. doi:10.1088/1748-9326/3/3/034002
6. EPA reports, 2008. Municipal Solid Waste Generation, Recycling, and Disposal in the United States. http://www.epa.gov/epawaste/nonhaz/municipal/index.htm
7. EPA reports, 2010. U.S. Greenhouse Gas Inventory Report.
8. Fargione, J. E., Plevin, R. J., and Hill, J. D. 2010. The Ecological Impact of Biofuels. Annual Review of Ecology, Evolution, and Systematics 41: 351-377
9. Forster, P. M., & J. M. Gregory (2006), The climate sensitivity and its components diagnosed from Earth Radiation Budget data, J. Climate, 19, 39-52
10. Hullu Maassen J. L. W., van Meel P. A., Shazad S., Vaessen J. M. P. 2008. Comparing different biogas upgrading techniques. TY/e report
11. Jiang, H., Chen, Y., Jiang, P., Zhang, C., Smith, T. J., Murrell, J. C., & Xing, X. H. (2010). Methanotrophs: Multifunctional bacteria with promising applications in environmental bioengineering. Biochem Eng J. 49. 277-288
12. Kaluzhnaya M. G., Khmelenina V. N., Eshinimaev B., Suzina N., Nikitin D., Solonin A., Lin J. L., McDonald I., Murrell C., & Yu. A. Trotsenko. 2001. Taxonomic characterization of new alkaliphilic and alkalitolerant methanotrophs from soda lakes of the Southeastern Transbaikal region and description of Methylomicrobium buryatense sp. nov. Syst Appl Microbiol. 24: 166-76
13. Kalyuzhnaya M. G., Khmelenina V., Eshinimaev B. T., Sorokin D. Yu., Fuse H., Lidstrom M. E., & Y. A. Trotsenko. 2008a. Reclassification and emended description of halo(alkali)philic and halo(alkali)tolerant methanotrophs of the genera Methylomicrobium and Methylobacter. J J Syst Evol Microbiol. 58: 591-6
14. Khmelenina V. N., Kalyuzhnaya M. G., Starostina N. G., Suzina N. E., & Yu. A. Trotsenko. 1997. Isolation and characterization of halotolerant alkaliphilic methanotrophic bacteria from Tuva soda lakes. Current Microbiol. 35: 257-61
15. Klan S., and Hagemann M. 2011. Compatible solute biosynthesis in cyanobacteria. Environ Microbiol. 13, 551-62.
16. Krich K., Augenstein D., Batmale, J. P., Benemann, J., Rutledge B., Salour D. 2005. Biomethane from dairy waste. A Sourcebook for the Production and Use of Renewable Natural Gas in California.
17. Laemmli, U. K., 1970. Cleavage of structural proteins during the assembly of the head of bacteriophage T4. Nature. 227, 680-685.
18. Lunn, J. E., Ashton, A. R., Hatch, M. D., Heldt, H. W., 2000. Purification, molecular cloning, and sequence analysis of sucrose-6-phosphate phosphohydrolase from plants. Proc. Natl. Acad. Sci. USA. 97, 12914-12919.
19. Lunn, J. E., 2002. Evolution of sucrose synthesis. Plant. Physiol. 128, 1490-1500.
20. Page-Sharp, M., Behm, C. A, Smith, G. D., 1999. Involvement of the compatible solutes trehalose and sucrose in the response to salt stress of a cyanobacterial Scytonema species isolated from desert soils. Biochim Biophys. Acta. 1472, 519-528.
21. Piccot, S. D., Beck, L., Srinivasan, S., Kersteter, S. L. 1996. Global methane emissions from minor anthropogenic sources and biofuel combustion in residential stoves. JCR Atmospheres, 101: 22757-22766
22. Sambrook, J., Russell, D. W., 2001. Molecular Cloning: a Laboratory Manual, third ed. Cold Spring Harbor Laboratory, New-York.
23. Schahczenski J., & Hill, H. 2009. Agriculture, climate change and carbon sequestration. ATTRA.
24. Scheehle E. A.& Doom, M. J. 2001. Improvements to the U.S. wastewater methane and nitrous oxide emissions estimates. Working paper. Washington, D.C.
25. Scheutz C., P. Kjeldsen, J. E. Bogner, A. De Vissche, J. Gebert, H. A. Hilger, M. Huber-Humer, & K. Spokas. 2009. Microbial methane oxidation processes and technologies for mitigation of landfill gas emissions. Waste Management &Research. 27(5): 409-455
26. Shindell D., Kuylenstierna J. C. I., Vignati E., Dingenen R., Amann M., Klimont Z., Anenberg S., Muller N. et al. 2012. Simultaneously mitigating near-term climate change and improving human health and food security. Science 335(6065): 183-189
27. Trotsenko Y A, & N. Khmelenina. 2008. Extremophilic Methanotrophs. Pushchino: ONTI PNTs RAN, 206 pp
28. World Bank and The Global Gas Flaring Reduction partnership (GGFR) 2008. Guidelines on Flare and Vent Measurement. By Clearstone Engineering Ltd. Tech. report.
29. Wuebbler D. J. & K. Hayhoe. 2002. Atmospheric methane and global change. Earth. Sci. Rev. 57: 177-210

### Example 2

### Dry Fermentation for Methane Biocatalysis

The mass transfer of gaseous O₂ and methane to the liquid phase culture represents a significant challenge to the overall efficiency of methane biocatalysis. One of the newly emerging technical solutions to increase mass-transfer is to immobilize cells at the gas/liquid boundary. This is currently under development as part of the large ARPA-e REMOTE program. A similar type of bioreactor has shown great potential for hydrogen production (Gosse et al., 2007). However, systems with immobilized methanotrophic cells, which have extensively been tested for methane mitigation, were found to be quite expensive and to clog easily (Yoon et al., 2009). While very promising, the area of methane catalysis needs new innovative developments which can eliminate clogging and contamination issues and couple methane utilization to the production of value-added compounds. Halophilic microbes can tolerate very low water activity (high salt or dryness) via accumulation of osmoprotectors (Khmelenina et al., 1999). It should be kept in mind that methanotrophs produce significant amounts of water via methane oxidation:

CH₄+O₂=H₂O+(CH₂O).

Thus, per 1 metric ton of methane oxidized 1.125 L of water is produced.

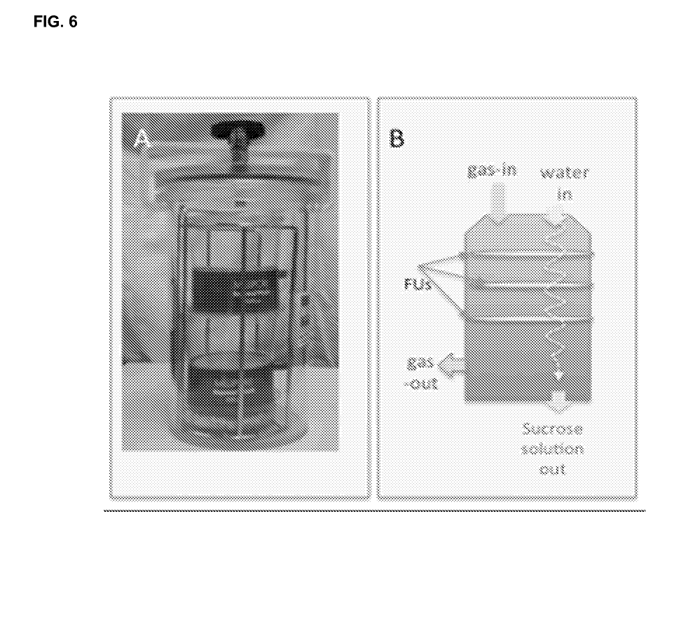

The inventors found that the methane-derived water can support core cellular functions and methanotrophs can stay active without external water supply for prolonged time. Cells of M. alcaliphilum 20Z were grown in bioreactor cultures up to OD₆₀₀=5, collected on 0.22 μm filters (250 ml per filter, or 0.25 g cell dry weight (CDW) per filter), air-dried, placed into incubation jars containing 50% CH₄ and air, and incubated for 3 months. Jars were refilled with 50% CH₄ and 50% air on a weekly basis (FIG. 6). It was found that approximately half of the added methane was consumed every week, which corresponds to 0.3 g CH₄ g⁻¹ CDW d⁻¹. The consumption data are significantly underestimated due a technical limitation of the experimental set-up. However, the data demonstrate that immobilized air-dry methanotrophic cells can stay metabolically active for at least three months. Thus, the unique properties of halophilic methanotrophs to stay active in immobilized stage without water supplementation provide a new and potentially transformative opportunity for methane fermentation. This new concept of methane biocatalysis is named Dry Fermentation (DR).

Dry conditions naturally stimulate accumulation of osmoprotectors (sucrose), and thus Dry Fermentation can be applied for methane-to-sucrose conversion. The sucrose can be extracted with water. Other sensitive parameters are: (1) flow rate. A high gas flow that is too high might dry cells quickly, however, a slow gas rate that is too slow might not provide sufficient amounts of substrate needed to allow the cells to regenerate energy and water and stay active. One of ordinary skill in the art can identify an optimal flow rate for the methods described herein; (2) Cell load. While a high load of cells might tolerate dryness better, it can also cause sub-optimal consumption of methane or limit methane accessibility to all cells leading to cell lysis. Again, one of ordinary skill in the art can optimize the cell load to obtain the desired (e.g., maximal) conversion of methane-to-sucrose; (3) wash of nutrients. In order to support immobilized cells, key nutrients (nitrate, sulfate, phosphates) can be loaded after each cycle of sucrose collection or harvest. The optimal parameters (cell/nutrient load and gas-flow rate) for the lab-scale dry fermentation can be optimized further with respect to a specific design of the DR-module.

All references cited herein are hereby incorporated by reference in their entirety.

1. An engineered methanotrophic bacterium, the bacterium comprising at least one genetic alteration that increases flux through the sucrose biosynthesis pathway, thereby increasing the conversion of methane to sucrose.

2. The bacterium of claim 1, wherein the genetic alteration results in an increase in the activity of at least one of the metabolic enzymes selected from the group consisting of: methane monoxygenase, hexulose phosphate synthase, phospho-3-hexuloisomerase, and sucrose-phosphate synthase.

3. The bacterium of claim 2, wherein the genetic alteration is overexpression of at least one of the metabolic enzymes selected from the group consisting of: methane monoxygenase, hexulose phosphate synthase, phospho-3-hexuloisomerase, and sucrose-phosphate synthase.

4. The bacterium of claim 1, wherein the genetic alteration results in a decrease in the activity of at least one of the metabolic enzymes selected from the group consisting of: amylase, and glucose-1-phosphate adenyltransferase.

5. The bacterium of claim 4, wherein the genetic alteration is a mutation in the gene encoding at least one of the metabolic enzymes selected from the group consisting of: amylase, and glucose-1-phosphate adenyltransferase.

6. The bacterium of claim 1, further comprising at least one additional genetic alteration that
(i) reduces the activity of at least one of the metabolic enzymes selected from the group consisting of: gluconate-6-phosphate dehydrogenase, ADP-glucose pyrophosphorylase, glycogen synthase, and glycogen branching enzyme, and/or
(ii) increases the activity of methane monoxygenase.

7. The bacterium of claim 1, wherein the bacterium comprises between 0.1%-30% sucrose content.

8. The bacterium of claim 1, wherein the sucrose content is increased by at least 20% as compared to the corresponding wild-type bacterium exposed to the same conditions.

9. The bacterium of claim 1, wherein the bacterium is selected from the group consisting of: 20Z, 5 GB 1, Methylobacter bovis, Methylomonas sp. LW13, Methylomonas MK1, Methylomicorbium buryatense 5G, Metholobacter luteus. Methylocaldum szegediense, Methylobacter marinus, Methylobactertundripaludum, Methyloglobulus morosus KoM1, Methylohalobius crimeensis, Methylomicrobium album, Methylomonas methanica, Methylosarcina fibrate, Methylosarcina lacus LW14, Methylobacter sp. 31-32, Methylovulum miyakonense strain HT12, Methylococcus capsulatus Bath, Methylococcus capsulatus Texas, and Methylomonas sp.11b.

10. The bacterium of claim 1, further comprising an additional genetic alteration that reduces flux through the ectoine biosynthesis pathway.

11. The bacterium of claim 10, wherein the genetic alteration results in a decrease in the activity of at least one of the metabolic enzymes selected from the group consisting of: ect A, ectB, and ectC.

12. The bacterium of claim 1, wherein the bacterium comprises a carbon conversion efficiency of at least 45%.

13. (canceled)

14. A method for fixing methane carbon in sucrose, the method comprising contacting a bacterium of claim 1 with methane under conditions suitable for methane catabolism.

15. A method for performing dry fermentation of methane to sucrose, the method comprising: contacting bacteria as claimed in claim 1 with a gaseous mixture comprising methane in the absence of exogenously applied water, thereby dry fermenting methane to sucrose.

16. The method of claim 15, further comprising a step of removing metabolic water and/or sucrose produced by the bacteria during dry fermentation.

17. The method of claim 15, wherein the bacteria are immobilized on a solid support.

18. The method of claim 17, wherein the solid support comprises a filter or a polymer.

19. (canceled)

20. The method of claim 15, further comprising a step of introducing additional bacteria according to claim 1.

21. A method of removing methane from a gaseous waste stream, the method comprising contacting a gaseous waste stream comprising methane with bacteria as claimed in claim 1.

22. A methane fermentation bioreactor, comprising a plurality of solid supports comprising immobilized, viable, methanotrophic bacteria according to claim 1, the supports located in a chamber comprising a first inlet supplying a mixture of methane and air or oxygen, and a second inlet permitting periodic flushing of the solid supports with an aqueous composition to remove sucrose produced by the bacteria and a first outlet permitting collection of sucrose, wherein said supports are arranged and held in the gas phase during methane fermentation, and wherein the bacteria remain viable and metabolically active for fermentation using water they produce via methane fermentation, exogenous water not being necessary for viability or metabolic activity.

23.-41. (canceled)


SEQUENCE LISTING (18 sequences)

SEQ ID NO: 1 - protein, 6 aa, Artificial Sequence
  Description of Artificial Sequence Synthetic 6xHis tag
HHHHHH

SEQ ID NO: 2 - DNA, 33 bp, Artificial Sequence
  Description of Artificial Sequence Synthetic primer
ttagatctcggccaaaattggcgatgagttggt

SEQ ID NO: 3 - DNA, 34 bp, Artificial Sequence
  Description of Artificial Sequence Synthetic primer
taccatggacgatgacggcagccggcttatcgac

SEQ ID NO: 4 - DNA, 35 bp, Artificial Sequence
  Description of Artificial Sequence Synthetic primer
ttccgcgggcacactctataacctggatcagcatg

SEQ ID NO: 5 - DNA, 31 bp, Artificial Sequence
  Description of Artificial Sequence Synthetic primer
tagagctcgcctcatccgccttggtcagtac

SEQ ID NO: 6 - DNA, 28 bp, Artificial Sequence
  Description of Artificial Sequence Synthetic primer
gagacgtcgctcaaccgactcattgacg

SEQ ID NO: 7 - DNA, 28 bp, Artificial Sequence
  Description of Artificial Sequence Synthetic primer
gtcagctgggatgaatcggcctcattcg

SEQ ID NO: 8 - DNA, 28 bp, Artificial Sequence
  Description of Artificial Sequence Synthetic primer
gagggcccgtggaatagtagcgctaaac

SEQ ID NO: 9 - DNA, 33 bp, Artificial Sequence
  Description of Artificial Sequence Synthetic primer
gagagctcgatcaaaatccggcgtcgggggtcg

SEQ ID NO: 10 - DNA, 32 bp, Artificial Sequence
  Description of Artificial Sequence Synthetic primer
gaggggcccgtggaatagtagtgctaaacaat

SEQ ID NO: 11 - DNA, 33 bp, Artificial Sequence
  Description of Artificial Sequence Synthetic primer
gaggagctccgtcaaaggacgccgtgagcccag

SEQ ID NO: 12 - DNA, 31 bp, Artificial Sequence
  Description of Artificial Sequence Synthetic primer
gaggacgtccacagcggctttgactggatcg

SEQ ID NO: 13 - DNA, 33 bp, Artificial Sequence
  Description of Artificial Sequence Synthetic primer
gagggtaccgagtttaccgaggtggatttcgcc

SEQ ID NO: 14 - DNA, 29 bp, Artificial Sequence
  Description of Artificial Sequence Synthetic primer
gaccatggcacaacggcatattggattgc

SEQ ID NO: 15 - DNA, 27 bp, Artificial Sequence
  Description of Artificial Sequence Synthetic primer
gagaattcgctgtcggcatctttgatc

SEQ ID NO: 16 - DNA, 18991 bp, Methylomicrobium alcaliphilum
gctcaaccgactcattgacgaaaatttcgatgacattcaaaaaattatcgccggctaccaggtccaacttttaccgatcccaaaacaaaaaagccgcaaagacgacgactaattcatcggatacattatttcacttggagcagtcaagcattcggttttatcctaagttcggtagtttaaccatgaagctcatgaaggacttcaaaaaattaccttaacattctcgctaaggaacgagcatgaaataacttcgacaaatgctgaaagggggttcggtggctcgattgacaggcgtcggcggcagggcagatttttgctcctgcaaaatccgcattcatgccatccatggcaatctgatagccgccgtcgagcctacatggatgtattcacccagcacctaaattccatagcccattggctatggttaactatcttggataatttaggtgctgggttcacggcgtcctgtcaagcgagtcgccgcaccaccacaatgcctaaaacttgtagaagtaaatttgtgcatattcctaaccttgtactctacacaaacgcagaacaccttattcgtcataatcttcatggtgaaatgcttttttctaggcttatagttgaggcggtaatcaaaatacggatataacatccaccaggctaaatcatttgaatttagcctggttttttcaggattaattctttttgggaggcgtcaacaccgcttgcggaccgggtgccgtttggctgtgttttaagacgacccctcctaaaggcggcaaggtgatatagagcgaatgaggccgattcatccaagcctgctcttgagtccaaataccgtccggcgaattaaccaccccgctgccgccgaattcctgcgcatcggagttaaggatttcgtcataacgcccagcagccggtacaccaatgtgataatactcccgcgggaccggagtaaagttcagcgcgacaaccaaaaactctcctgtatcggcagccttgcggatataaatcagcaccgagttatgggcatcatggcaatcgatccactcaaatcccgcatcggtaaaatcattggcatgtaaagccgtctccttttgataggtgtgattcagcgtcttaaccaaatgctgcagtccttggtggaaagggtaggccaacacataccaatcaagcacttgactgctgtcccattcgcgcccttgaccgaattcacaacccatgaagagcaactttttgccggggtgggtaaacatataaaaatacaccaaacgcaaattagcaaagcgctgccactcatcgccgggcattttatataacatcgattgcttgccgtgaaccacttcatcgtgagaaaacggcaacaaaaaattttccgtaaaggcataaatcaggctaaaggttaattctccctgatggtattggcgaaatatcgactctttttccatatagcgcaacgtatcgttcatccagcccatattccactttaaatcgaagcccaagccaccgacatacggaggcttggtaacctgcggccaagcggtcgactcttcggccatgattaacacgccaggaacttgctgatgggtaatttcgttcaatgatcttaaaaagtcgatggcctccaaattttcgcggccgccgtacttattgggtatccactcgccttcctcacgggaataatcaagatacaacattgacgctaccgcatcgactcgcaaaccgtccacatgatattcctcgagccagaatagcgcactcgagatcaaaaagttcttaacttcatagcggccgaaattaaagatcaacgtcgaccaatcacgatgttcgccgagccttggatcttcatgctcataaagcgatgtaccgtcgaaccatgcaagcgcatgcgcatcttttggaaaatgcgccggcacccaatcgagaatgacaccgatctcgtgctgatgacaataatcgacaaaccagcgaaaatcttcggggctaccgaaacggctggttggcgcatagtaccccgtcgtttggtagccccacgatttatcatagggatgctcagtaaccggtaacaactcaagatgagtaaagcctaaggttttggcatattccaccaaggccaccgccaattgccggtaattgagcaactcgccttcggagcctcgctgccaagaccccaaatgcacctcgtagatgctaatcggcttgtgctgccaatcgaacttctcacgctgtttcaaccatttttggtcatgccaattaaaattatcgtttgccgttacaatactggccgtattcgggcgaacctcataaaaccgaccgtaaggatctgtttttaataaaatatcgccattcttcgcgcgaatttcatacttataaaccaagccgggctctaagcctggaataaaaagctcccaaacgccattaccgcgcacccgcatttgatggcaacggccgtcccagcggttgaaatcgccgactacgctgactcgcgatgcattaggcgcccacacggaaaacagcacgccggcaacattttcgatttcgtgtacgcgtgcacccagcaaacggtaagcatgccaatgtttaccttcactgaataaatgtaaatcgtaatccgataattgcggcccgtagcaataaggatcgtaaccgatgtgctcgtaatggtcttgatcgcgccaaatcagccgatagcatggaggaacttgtgatgctaagccatgccattcgaaaagagccgtaccctcgattcgagtcaatggcaagtttccttcagccaaggccacggtttcggcatggggcaaaaaagctcgtaccacaacctgtttgtcatccttgggatggcggcctaaaacggcaaagggatcgctgcattttacatcgacaagactctgtaattctgtggacagttttgttttactcatctgcttttgcgggaagcccgcgaatttataccccttacttgtcaaatttcggtgccggggtgcccgtcaaaggacgccgtaaatacgtccatgtaggctctatgatagcatccatgctatcaaagcctttgccgaacaccccgacacctccatagactaatgccgaaatttaaagtgcgaaaggtggtatatcattcgggtggggatagctcagctcgcctcggcaagcccggttcccgctctcctgttttaatgtgctatcctggttttagcattgattcggcgtcgttattctggcacctgtaattgtaaaatcttaacttttaccgagccgtctactgtgtagtgaccatgacccgcaccatacccggcaataagtcgaaatgatagggactcggcacataagctagcggatactccggcgatagatcggtaaacggcgcgcgcgactgaacatattgatatagatcgggcgtcgaaaactcatggcgttgccaagggtctttatttaacaagatcaaagcttcttgttgtccggataggctggctttccagaaaaagagtatcggcgactgcggataaccgattaggttaaaaggcccctcttcctgaaatacagggtaatcggctttcaccgtgttgacttgagtaataaactcggtcaaatccacctcggtattttcccagtcctcggggcgagtatgcaccacatgcagcggtttactaaaaccgtattcgtaacccatcggcatcatcacgccgctgctgaacactgcggaaaacaagtaacgctgctttagggcttcggcattgccgttgctttcggcaaataaacgaggcgtatcgtgactttcgggaaaactgatcgacggggcggcttggcgcaataattgatactgttccggtagccaagggctttcgaagtcccaccatttggaactattgaaaatataatcgaacccggcttgcgcggtttgcttggttcgctcggcgctacagccaagcgtttcagcggtaaacaccacatcgggatgtttttgtttcgtatccgtaatcaattgccgccaaagttcgggcgctaattgataggcggcatcgcaacgaaagcctttaaaacccagatcgatgtatgactcgatgattttacgataataggcataaagcccattatcgtgactgcggccatgatcgagttgcgctaaatcctcccataccactttatggccgccgtcttcgagacaaaaaggacgctgcacctgcccatgctcacgaacaaaccattcagggtgctgttttaacaagggcgaatcgatcgcacagtgattcaataccaaatcggtcataaagcggagtcggtgttttttctcagcctgttgaatcaagccccgcaattgttgctcggcggtctttttggatttcggatcgactaacagcgggtttaactgaaaataatcggcaatagaatatagactgccggatgccccggttttttgtaacgggttgacgaaaacccaatcgaaccgcatcgccgcggccctttccaagtgcgcctcccactgcccgaagtggcctgccaacagggggaacaagttgtacatcctcatgaatcggcctctttatttgttacgaatatagtcatagatattcaggtaatgttggcccggatatttccaggagtaatcgtaacgcattgcatttttcatcaactcccgaaaatgatccggaaattgataataacaatcgatcgcgcgcgacaatgccgactccagcccttcattgttgtaatccctaaaaacatagccgttgcgctcgtgcaaggctttatgatgcgcatgatccttatcgaataccgtatcggccaatcctccgacctcgcgaacgatcggaaccgtgccgtatttcatcgatatcaactgcgtcaaaccgcaaggttcgaataaactaggcacgacgatcatatccgcaccggcataaatcagatgcgccaagccttcgtcgaaaccgacttcgatatggcaatcgggactgtcgttcaaatgccgcttcaactcccagaaatagctattgatatcccgctcgggacttgagcctagcaacacgaactgcgcccgatggttcaatgaataaaacaatgcatgacgaaccaagtccaaccctttctgaggatcgagccgcccgacgaaagcaataatcggcttttcattatccgctagcaacaagcgatcgcgtaaagcccgtttattgagatatttattgtctactttttccacgataaagggagcggcgatatgcgggtcgatttcgggattccagacatcataatcgatcccgttgaccacaccgccgaacttataatgatgcgcatgaagggtcggttccaaaccgaatccttgaccgccgtctttggtttctccggcataacgcggcgataccgtcgtaacgaagttggaatagactatacctcctttcatcaaattcaacgcatgcggaatccgattatccaacaagcgatcatcatgaaaataatactccggtcgatgcaggcccgtcgcattcaataagaagtcgcccgtcaacccttgatgcttgaagttatgaatcgtaaaacatactctcggatgccagaagcctaatttctgatagatctcatataaaaacacaggcaccaatgccgtttgccaatcatggcaatgaatcacatcgggatgcttgccggatttccaaagaaattccaatgcggcacggcaaaagaacgcataacgcagcacgtcgtcattaaagccgtaaatcgtgccgcgattgaagaaattgtccggggaatgcggctcgatgaaaaagcatttacggccatgaacgaaaccgaaccagaccgtacaatgaatatggccgttataccagggcacccaaaggtcgtcataggttttacataagccccaaatatgatcgtaatgcatacaatcgtatttgggcaaaataatctcgacactgtgtccgcgcaattccagttcgcggcttaaaccggtcacaacatccgccaaacctccgaccttcgccaagggcgccaattcaggagtaatctgcacgacaaacatatgcgggtagtcgggaccgggttggtcatggtacggcgaatgctcgacgtgcgacatcatgtcggcaacgggctccggcgccgaagactcgggtgtcggcgctattggctctgccttactataatagggttgccgctgaaacttttccgacggcacatcgggactgatggatgccgcctgagcaacctctggcaaaggcgtgataaatgccgtattgggctcccggtatacggattcgccgcccttggaagacgtatcgacttgggtttcgccggcggtaccggactgtttaggaacggctttttcggaagcttgactaggtgcagccgccaccttcgaagcaacattctctgccgccgctgtctcgttttgcggaacaacgggtaaaaccggaacaggcgacaacgactccgcttcggatttttcctgcgccgttttttcggatgttttaatctgccgatcggatttagtctgcgtatctgatattttgctcgctgaattacgcattagcgccgacttaagttcccctgcagacttagttttcggagaatccttagcttctatttcggctttagccttctcttcatccttcttagtttgagtcggcgccttgacgctagatgcggtcttggttttttcggcaagcttttttttgggagcggctttcgccttaggcttaacgggggattttgccgtattcgcagtttttgacgaagcctttttcacaggctctgcttgagccggttttgcagcctgcgctttgggtgaagatttaacttccggcgccgttttagctttcggaactgttttaggttttgctgccgtttcggattccgaagacaggctaggcttcggcgcatcatcggtctgagtcgacttttccattttggaagattcggctttagggtcgttgttaaccgtcgacgcggcagcgctcggctgctcttccgcctcaggcgttaccgcggattgattctctgtcgtcggcttttcggacgccttattcgttgttgctgtttttgttgagcttctggttttagttgtagtttgctttgccataaatcagacctatcatgccaattaaaagatatactttcccgtcgcccttagtatttcgtgacaaataacgtccaggaactatgagccttgttgagggctcggtaacttgtttaacaggactctctgccgtcgacatctatcaagcgaccaaccaaacccgcctatcctacattaaaattagggaagttttatgaccgaaaccttagcttagtatgttgtccgcagttgtgtttttataagtccgaaaatcaagcatcggaaaaatatcatccatcacttctaacgccgtcaaatagtgttcgtttattgtatccgtacgaacgctttcttgcagataattaaatcgagccaaatgatcaagaatacgctttttagcatactcgacggtcgttcctgatttcataatgaacggccaatccgaggcctgcgccaataataacgaccgtgccgcctgattcaaagcccgctcctccaaggaaaaacggggagcttgctgaaactcttcggctaactcctccatcaacatcgccgatttgtgtaaaacgggataaatccaagcattggtttcattgagccagtatccggaatagccctgttcgccccaactcgatgccgaaggcgttgcggtttgcaagttttgaaaacgcgacaaataatcgctaccactgaccgactgcattcccaacttattgccgccaaccaggcgtaataaaaactccagccattgcggtccctcgaaccaccaatgaccaaacaattcagcgtcgtagggagctacaatgattggcgatttcctcatccgatagctcaggtcgtcaagctgccgttgccgcttcaaataaaagtcttgggcatgttgcctcgccttcatcaatgcttgacgcggccgataggccgctttcgggccaccccctcccgtcactcgctgatatttgatgccgacgggtacgcgaatcgtttcttccaataaatacggagcgagataactctccggaatggtggaatcataaccgatatcgcaatgatattcacgatagtcgccgtctcccggatacccttcctgcgcgctccagacttgacgcgacgattcgggatcgcgggcaaaagccgccacaccgttagtacaggctataggcgcatagacgccattttctgattgcgggcttgcttgagtcaaaccatggctatcgacaaaaaaataacgaagatcggcttcgtcaagcaacgtctccaagcccggataataaccgcattcgggcaaccaaaatccttttggcataaaaccgaagcactgtttgaaagtttcaacgcccatgttaatctgattgcgcaccgcactttcggaaacgttcagtaacggcaaaaaaccgtgtgtggccgcgcaggtaatcaattccaggccgcctaattccgcctgttttttgaaagccgcaactaaatcgcaatcataatcctcccgataacgctgcaatgcggccttgaaattacgtcgatataaacgagccaaatactgatattcagcttgcttgcgagtccttgaaacttcctgctcggctaatctgatcaaaccttgcaaatggcgaacatagcgctgctgcaataactcatcattcaacatcgtcaacaaggtcggcgatagcgacagtgtcagtcgatatcgcacgcgatcccgattcaagcgctcgcacatgctgagcaagggcaaataacactcggttatcgcttcgaataaccagcgctcttcaaaaaaatgctcatactcgggatgatgtacgtaaggcaaatgggcatgtaatacaatgcataaaccgcccctatccatctcaagtacctaaggaacgagcatgaaataatttcgacaacagttgaaaggagggctggtcggcaattcccagtttggggatcaaaaagagtgaggaggatgcttggcgaggacttcggctgcagggagcagccgtcaagcctccatgaatgggtttacgacatcctgtcaagcgagccgccgaaccgacgcaaagcctactacttgtaagagttattttgtgcatattcttaagtgagttttattgctattctgcagacaggatataccggcactcgaataactatccatatcatggagctcaaatcgttctgctgaggtcaagtgaatcggagacgccggtgaaaacgggatatcttcggcatccggcttgctcaatgtttgccgaattttagcgtcaatatcggcttcattaaaggctaaaggccttgttagcgcggtcaccggcgtcatcgcagaagaaggtatactaacaggctgcgagcgaacatatacaataaagtccccattactgtcgcattgaccaatcgccgccgtataaattgcaccggcaaccggcaattgaattttagcattggcgtgcgtggataccacgccgacatcaaaccatggacgctgatgctcttgagtatccgcccaatcaacctgccagaacaagcgtaatattaacggattattcgtcgcctgcagcagaggagtaacgccggcaatacgccaataggcatgaagatgatagggatctatcggtaaaataataagctgtggaaaattaagcggttcagtctgactgaattgcaaaggatgataggtatgactaatggtttgactgatgtgttgaatttcttgagacgatatagtgagcttagggttatgcctggaatgccaatatctcattcattttttcctgatttcataaatcttttaaccacattcactgcatttgaagggtaaccgtcatatcggcatgatccataggcggattatcctgactgaaaccttaaagtgtcaattcaaattaggctttgccaaccagattttggcggaagacaacccaagcaaaccttcggcaacctttgcccagcttaatctcgcccgtataatttgatcgcacgcgccaaacgctgactgtgcaatggtaataattgatcccaagtaaaacgccacaaccaattgccttcagtcgtaccgggaatattcatgcgatgctcggtatctaactcaagaatgtcctgcatcggaatgatcgcgagattcgcaaccgaagctaaagcggtttgtattagcgtatagggcatcgagctagcaggctgacctaagtattcataaacataagcttgctcttgctcactgatctgacggtaccatcctaaggtagtgtcgttatcgtgcgtgccggtataaacgacgctgtttttatccagattggccggtaaataaggattactggggccgccgctgaacgcgaattgcagaatcttcatgcccggcaaagagaaatcgtccctgagtttttcgactttatcggtaatcacgcctaaatcttccgcgaccaatgcaataggtccgaacgcatcgaaaaccgtctttaacaaggcgtgccccggcgccttgacccagcaaccattcattgccgtgggttcggataccggaatctcccaggcggcctccaaccccctgaagtgatcgatacgaataatatcgaacatttcgacttgcgtcttgattcgatcaagccaccaatggaagccattttttttaagatatgaccagttgtaatgcggattaccccaacgctgccccgtttccgaaaaataatcggggggtacccccgccactaccgacatttcgaactgctcatccagtttaaacacctccggatgaacccaaacatcggcgctatcgtatgaaacgaaaataggaatatcgccaaacaacaatacaccatggcgcgccgcataatctttaagttcttgccactgtgtaaagaatacgaattgctcgaatttaatcgcttcaatctcagtgcttaggcgtctggaggcttctttaagcgccgaaatattgcgcgatttataccgttcaggccattgattccagctaagattattgaattcttttctgagtgctataaataaggcgtaatcatccagccaaaactttttgtcgatacaaaatttggtaaattcgattttttcgtgttcggtcgcgtgcagcttaaagccataatatgcgtgattgagtaaacactcccttgcaaatgacggactgccgccacaatcttgacaacgctcgccacggctcagccagcctttgtcaaccaacttatccaagtcgatcaaagcggtattaccggcatgagcggagatgcactgatagggagaaccatcatcatgaggcatgtttaatggcaaagtttgccaaaccgttacaccgacactatttagaaagtcgacgaaccgataagcttccgggccgaaattaccattatcacgattgcctggaagtgaagtaatgtgcaataaaattccggcacggcgtctatgtaagatatcaatcacaaacgtctttcctgactaaacgttttcaatacccggcctcatcgtaccgcccatggcgggagctccggacccctgagtgaacgataatgccagataagccggcggatcttctcctaacaacagatagagattactcaagttcaaacgaaactgtttttcaaagctgctgacagcctcccccgggttgtaatcgccgaaccaccaaaaccaatccgaaccttcacaaatagcaagctgatgctcggcaagagccaactgctcctcggtcaagcgcccggtcgacacgactttatcaaacgcccatttagcatcgcccagcatatcccaaccccggtttttatcgacatcgccgatccaggtagaaaacgttccatagacccaactgccagccatcaaattcggaagcgtttgaatttcaatgacttgatcgaggcattcggaaaatgttgttagctgaatctcggggtgatgactcaagcgtttataaagcgcgctgagaaagtgataaccgttttgcggaaaatactcccaagcattttcgccatccatgataatcgataccaccttatccgtagaagtatcatgcttatggatttgttccaagtgattgaccaaatcggcaaccgcatcgtcggcatgccatttggaatattcaaatccgatcaaatccgataagccatcgtccctaaaaaaacaaggtataccggttccgctagcgcgaaacggatgatggggactcacgctatcgatgcctgataatcgcaagctgttatgcaagacattaccgcctgttgcagcccaatcgaaaccgaactcggagagcatctttaatgtctcggtactgactgctccctcggatggccaacagcctctcggcgtgaatccgaaaaatcgtttaaaggtttcgacaccatgcttcagatgccatttcaccctttcctcgccccccggataatgatccagttcgggtagcggagcatccggcatcgcctgcaatgtacttttaatatcgattaacaaaggcataatcggatgcgcataaggcgttaccgaaagctcgatgcgtccttttcttgccaacgccttatatcgacatattgcagtggataattgctcaccgatgacctcgacgatttcgatccgttcatgcaatgtgtagcctgaacctttggcaatcaagcgcttgaccctgacatcggtcagcttgacactctcgcccatccaagccagatgataccacgctaaaagatcgctgatgaattgagaattcatatagctcaacgcatcatgattctgctccatccattcggccattttgaccagtttctggaaagtcggatatcgtccgatctgccgctctcgattggcacgcatgcaatccttgaccagttttagccgtgcatcgggatccgagggaatcgatggggcaaccagcgccgccaacaacggatctttaaatgagatgcgatcatgcagatagcgccccacttgcctggcataatcctcgatctgctctagaaggatcggcgcaaaattaacgaccgctttggcttccggcgccgcttcaagatgggcgatcatgtcgacataatccttcataacatgcaaataggtccaaggcagttgaaattcgccggtttgcaaatcgcgatactcaggctgatgcatgtgccaacatatcaccagcttcagttttttatctgacatagtgcctactctgtcctaacatatccggggtgaccaatacgactccgcccgggctgacatgaaatctttttttatcgtcctcgaggttctcgccgattaccgtgccttggggcacttcgcaacctttctcaatgattgccttggtaattcggcaatgccttgcaatattgacctcaggaagcaccacgacatcttgtaacgtcgtataagaattaacccggacattggaaaataacaatgagtgcctgaccttggcgccggaaataacgcaaccgccggacaccatcgaatcgaccgcctgcccccttcttttgtcgtcgtcgaagacaaattttgccggcggagtttgttcctgataggtccaaatcggccaagttttatcgtataaattcaaatcgggcttgacgccgattaattccatatttgccgcccagtaggcatctatcgtacccacatcgcgccagtaactttgatcgccttgcaaattcaggaatgggtatgcactgaccctatatttgtcgataaccgcaggaataatgtccttgccgaaatccctgttcgagcctttgctgtcggcatctttgatcaactgctcgaataaaaatgccgcattaaatacatagatacccatcgaagccaaggctttgtccggcttacccggcatcgaaggcggattggcgggtttttcgacaaatgcttttacccgtcgattttgatcgacgtccatgaccccgaactcggtcgcctcttcaaggcttacctcgagacaaccaatggtcaagtccgcattattggccacatgatcggcaagcatttcgccgtagtccattttataaatatgatcgccggccaatatcagaacatactccggccgacgggttcttagaatatcgatattttgataaaccgcatcggcagtcccctcataccaagacgtttcgatgcgttgttgagccggcatcaaatcgacatattcaccgaactcgccgcgtaagaagccccagccttgttgaatatgtcgaatcaaagaatcggctttgtattgcgttaaaatgccgatttttctgattccggaattaatgcaattggataacggaaagtcgacaatcctgaattttccgccaaaaggaactgccggcttggcacgccaatcagtcatattttttaaccgtgagccgcggccgcccgcaagaatcaaggcaattgtatttctagttaaatgactgacgaacctgtcttgaggttggttaattgactctgacattttcattccccttctggaattgactattacatctgttgcttaggtggtaacattctgataaaaatcattctactacattgaggcgtttcgaagtgaaaaagcaatccaatatgccgttgtgtaatgatttatgcaaaatcattgaagccaaacatcatgaccctttttccgttctaggccgcctcagcgataacaacctgaccaaggtaagactctatttgccttacgccgattcggttcgttttgctgaaaaaggccctgaaatacctcgactcgcaaaaacggacttttttgaatataccgctcaggccggcgaattaccagaacattatcaaatttcttggatcgacaaggaaggccgacaacacaccgattacgacccttatgactttcccgagcaattgccggaattcgatcgtcatttgttcggcgaaggcaaacattggcatatctaccaaaaacttggcgcacatcgatataaggtcgacggcatcgatggcgtattatttaccgtctgggccccaagcgcggagcgcgtcagcgtggtcggcgattttaaccgctgggacgggcgctgtcacttaatgcgagtgctaggtaacagcggggtctgggagctttttattccgggcctcgacgcgggctgtttatataaattcgaaatactgaaccgtcaaagccaagaaattttgattaaaacagacccttatgcccagcaatatgagcatcgccctaaaaccgcttcgattgtcgtcaaagaaggagcctatcaatggcaagatgcgaaatggatgacttatcgcaaaacccgcgattggcttcacgaaccgatgtctatttacgaagtgcatttaggctcgtggcaacgggacgatgccggaaatttcttgaattaccgtgaactcgctaaacagttaatccaatacgtcaaagacatgggctttacccatatcgaattactgcctatcaccgagcatcctttcgatgcttcatggggttaccaatcgacgggctacttcgccccgaccagtcgacacggatcacccgacgatttcagatattttatcgatctctttcatcaaaacaacatcggcattattttggactgggttcccgcgcattttccgaaggactttttcgcactggcgcgattcgacggtagcgcgctctatgagcacgaagacccccgcaaaggcgagcatagagattggggcaccctgatctttaacttcgggcgcaacgaagtcaaaaatttcttactggcgagcgcgatattttggctcgaagaatttcaccttgacggtcttagggtcgacgccgtcgcatcgatgctttatctggattattcccgcgatgaaaacgactggattcccaacatgtatggcggcaacgaaaatctcgaagccatcgcttttttgcgcgaactcaataccgtaacgcatgaacaacaccccggcacggtcgtgatggcagaggaatcgaccgcatggcctcaggtaacccgcccgacttggaccggaggcctgggtttttcgataaaatggaacatgggctggatgcatgacattctcgattatataagccgcgacccgatacacaggcgttatcaccacgatcaactaaccttcggcctactttatgcctttaccgagaacttcgtccttccgttctctcatgatgaagtcattcacggcaaaggttcgttactcaacagaatgccgggagacgaatggcgaaaatttgcgaatttacgcctactttataccttgatgtttacctacccgggtaaaaaattattattcatgggttgcgagttcgggcaaggtacagagtggaatttcaaccaacccctggattggtatgtactgcagtatcctcaacaccaaggtttgcaaactttggtcaaagatcttaatcatttgtataaaaactacccggcacttcaccaatacgacttcaatcacagcggctttgactggatcgattgccatgatgtcgaacaatcgattattagctaccgccgcaaaggcaccaacgatgatttgatcatcatacttaacttcacaccgattgtgagagaaaactatcatatcggcgtgccttccgaaggggtttattttgaaatctttaattcagactctgcctattatgaaggtagcaacatcggcaaccgcgagatactatcggaaccggagccttggatgggacaccaacaatcgatacacttgactttacctccgctaggcggaatcatcttgacgaggcaaacgaaagccataagcagctctgctcactaaatagcgtcctgtttgagaaagatgatccagcgtggcattgctcaccgcaaattaactggcgccggcctagcttataaaaaattcacttcgatatttatacgtgataggcacaactacgcgagaaatgcccgcaaaatagaactgccgccgccccgaacgcaaacttaaactgattcttcaataacaccggacgatcctacaacagcagacagtacgccaataaacactcaatcggctattgtggcgaaatccacctcggtaaactcaattctttaatatatacctagaaccgaattttgactgcctttaaatttaataattatgaaacgaattctttttgttaccagtgaagcacatcctttaataaaaaccggcggcttggcagatgtttcaagcagcttacctaaggctttggcggatctaggccaggatatccgtatcatcatacccaactatcaagctataaaaaaaaccgaaaacgtccaacatcggtgtacgttgagaattaataattgcgatgtcaatattctcgaaacccgcttgccggaatcaaaagtgatcgtatggctgatcgattgcccccagttttttggctacccgggcaatccttatcacgatgaatacggtaatgcctgggcaaacagcgccgatcgcttttcgctgttctgccgcataacagtggaagtcgcgatgaatagagcctacttagattggaaaccggagatcgtccactgcaacgactggcaaagcggcctagttcccgccttgttaacgctggaatacaatcgcccggcaaccatttttacgattcataacatggcctatcaagggatttttccctattcgacctacaatgcgcttaatcttccaagacaactttggaacccaaatgcacttgagtattacggcaacatgtcgtttttaaaaggcggcattgcttgctccgatcgagtaacaacggtaagtcctacctatgccaaagaaattcaatcatccgagtttggttacgggctagaaggcttgttaacccatcgcaaggaatttctaagcggcatactcaacggaaccgacaaagactggaatcccgaattcgacagcaacatcgtccagcgctacagccataaaacgctccatcacaaacaaaccaataaggccgcattacaagaacggctctcactgcctctcgagccgtcgattcccattttcggattaattagccgtttggttgaacaaaaaggtatcgacttgctattagaatgcttgccggaattattaagtatgccagtacaatttgtattactcggtagcggcaacaaaagcttcgaacaacgtttatataattttgccgaagcctaccctgaaaaaatgtcgattactatcggctacgatgaacaattggctcattggatagaagcgggaagcgatattttcttaatgccatcgcgcttcgagccctgcggattaaaccaaatgtatagtcaacgctatggaactttgccgatcgtaaggaaaaccggcggcctagcagatactgtcgtcgatgctctaccccatacgattgacaacaaaacggcaaccggaattacttttaatgaatcgacccccagttcgctactggaggccatcaagcgcgcgttgattttatacagagtcccggaaatttggacacaactgcaaaccaatgccatgaagaaagacttttcgtggaatagtagcgctaaacaatacttatcgctctatgaccatttgtaactcgattaaggatttggtcgtaaataacttccctgttttatggagagttcggtaactcgattggcaggtgtcggcggcagggcagattttttgctcctgcaaaatctgcattcatgccatccatggcaatcagtcgccgcggtcaagcctacacggacgtattcacggcgtcctgcaaagcgagttaccgaaccctcaacaaagctcatagttccaagacgttatttatcacgaaatcctaaggcgggctataactccaaaaagagttatttatcttttaccatgaagagcatgaaggacttgaagatacagcatttacaattaaacgtaagtgactgtccattaacagcctatgtcatcattattgccacttcagtctcgccattgttttgatattttttagtcattatacctttcgcacttcaaatttcggcagtgcctaaagagacactgggtccctctcctaacgctaggtgagaagctgggctcacgacgtcctttgacgacccccgacgccggattttgatc

SEQ ID NO: 17 - DNA, 2121 bp, Methylomicrobium alcaliphilum
atgacgagcaagaaggtttcgcattatattcttttgatcagtgttcacggcctgatacgcggacgagatttagaactgggacgcgatgccgataccggaggtcagaccaagtatgtcgtggatttagcgagggctttagcttatcaaccttcggtaggtcgggttgatttagcaacgcgattggttgacgatcccgaggttggtgcagattatcgcgaagcgcttgagccgctggataaaagcgcgcaaatcgtccggattccggccggccccgaaggctatatcaagaaagaggagctttgggattatctcgatattttcgccgacaatttgttggagtggctccggcagcagacgcgtatgccggacgtgattcatagccattatgccgacgccggctatgttggtgtgcggttgtcgcttttaaccggtatcccattggtgcataccgggcattcactgggcagagacaagctcggtcgtttgcttgcaatgggcttatcgagtgatttgatcgagcagcgttatcatatttccaaaagaatcagcgccgaagaggatgtgctggccaatgccgaattggtcgtgaccagtacccgcaacgagattgccgaacaatacgagctctacgactattatcagccggaacgcatggtcgtgattccgccgggaaccgatctggagcagtttcatcctcctgaaaacaaggtaaaaatagccttcggcaaatcgctggagacttttttgaataatcctaaaaaaccgatgattctggctttgtcgcgtcctgacgaacgcaagaacattgtttcgttagttcatgcttacggcgagtcccccgaattacagaaattagcgaatttagtgatcgtagccggtaatcgcgacgatatccgggaaatggacgaaggcgcgcaagcggtgttgaccgaaattctattattggtcgattactatgatttatacggccatattgcgatccctaagcaccacaaacaggaggatgttccggatatctatcgcttggccgcgctatcgaaaggcgtgttcgtcaatccggcattgaccgagccgttcggtttgactttactcgaggcggccgcatgcggattgccgctggtcgcgaccgaaaacggcggcccggtcgatattatcggtgcttgccataacggtattctcgtcgatccgctagatagcagtgcgatagcgaatgcgttgctggaaattttaagtagcccgaaaaaatgggaaaaattttccgaaaaaggccttaaaaatgttcggaaacgctattcctggaatacgcatgcacaaaagtatcttgagacgatacagccattattggagcatcatccggagtttccgaaagtggcgcgcgtgcgaggcgatttgagctatcgcgatcgagccgttttttccgatatcgatcaaagtttattgggcagcgccgaaggtttaaaagcatttgctgcttatttggccgagaatcgaaaaaaagtttttttcggtgtcgcgaccggccgcaggctcgattcggcaatggcaattttaaagagaaacggcctgccggtaccggatgttttaatcaccagcttgggaaccgaaattgtctattcgccgcaaatgacgccggacagcgcttgggcaagacatatcgattatcactggaatagcaaggccatacgccgcattatcggggaattgcccggtttgactttacagcccaaaagcgagcaaagccgatttaagatttcctatcattacgatgccgagcaggcgcctacggtagaagaaattcagacattgttgcgtcaggaggagcaggcggtcaatgtgtatatatccttcgggcaatttttcgacatcgttccggtgcgtgcatcgaaaggtttggccttgcgttatttcgcccagcagtggaatctgccgttggagcgtattctagtcgccggcggttccggcgccgatgaagacatgatgcagggtaataccttggcggttgtcgtcgctaatcggcatcgcgaggaattgtcgggtttgcgcgagcccgagaggatttattttgcggcgcaaccctttgcgctcggtatcatcgaggcaatagaacattatgatttttaa

SEQ ID NO: 18 - DNA, 1956 bp, Methylomicrobium alcaliphilum
atgtacgaacagatttcgcattccttattaaatgaaattttagatgatctcaagccggaaatccgcaggcaggatttgcggcatttttacacgcgtctcggcgcgaatttttatgcgattcataatctgtttcatcatctatacggccgccgcgacgatttcaaagagcaaatgctcaagctggtcgaaaccttggctcgacaatatattcagcgaccggaagagttggaaaaactcgatatcgcgcgggaaaacgatcataactggtttctgaatcaaaaatgggtcggcatggctttgtatagcgccgggtttgccgacgacttggccggcgtggcggataatacctcttattttcaagagctcggcatcaatttggtgcacatcatgccgatactcgagtgtcctaccggtcgcagcgacggcggttatgcggtcagtaatttccggcagatcgatagccgtgtcggcaaattagaagatttgcagcgggtttcaagcgagttcagaaagcgcgatattctgttgactttggacgttgtgttgaatcatacctcggacgagcatgaatgggctcagcgggcgaaggcaggagagcaaaaatatcaagattattattacatcttcgataatcgtgaaattcccgatatgttcgagcaaagtatgcccgaaatttttccggaaaccgctccgggaaattttacctgggacgaaactttgcagaaatgggtaatgacggtcttcaatgattatcaatgggatctcaactacagcaacccttcggtatttattgaaatgctcgacgtgattctcttttggatcaatcaaggcgccgatattatccggctcgatgcggttgcatttttatggaagaagatgggcacgacttgtcaaaacgaacgcgaagcgcatctgttgctgcagttgatgaaagactgttgccaagtaacagcgccggggtctttgttcattgccgaagcgattgtcgcaccggtcgagattactaaatatttcggcgaggacgcgattatcgcgaaagagtgtgaaatcgcctataacgcgactttgatggccttgctttgggatgcgttggcgacgcgcaatagcaaattactgtatcagggcttgaaaagcctgcccgataaattggagcgagcgacttggttgaattatgtgcgttgccatgacgatatcgggctaggtttcgatgacaacgatgtgcggcatgtcggttatgatccggtcgcgcatcgtcgttttttggtcgatttctatacaggggcttatgaggattcgatcgcgagcggcgtgccgttcatgcgtaatgagaaaaccggcgatgcgcggattgccggatcgctggcttcgttggccggccttgagacggcgattcagtccgatgacgaacatgcgatcgatattgcgttgaacaaaatcatattaatgcatagcgtgatattttcattcggcggccttccgcttctgtattacggcgatgagatcgcgacctttaatgatcacagttatctcgatcaacccgataaggctaatgacagtcgctggattcaccggcctaagatcgattgggagcgggccgagcaacgtaagaaacaaggtacggtcgagtatgccgtatttagcgcattgaaaaaaatgatcgcgattcgcaaggaaaccacggcttttgccgacttcaataatcgacagttgatcgaatgcgaaaacccgcatattttttcgtttatccgttacgatcataaccggccttcggaacaagtgctggtgatttgtaatttcagcgaacaacctcagcgtatcaagatggaaaaatttaatacggcggggtttggcatttacggacaagtcagcgatttatacaccggcaagcgtccgactgaattcgaccagcaactggtcattcagccgtgccagttttattggttaactgagtattaa